(19) United States
(12) Patent Application Publication
(10) Pub. No.: US 2017/0304466 A1
(43) Pub. Date: Oct. 26, 2017

(54) AAV-Based Gene Therapy

(71) Applicant: Arthrogen B.V., Amsterdam (NL)

(72) Inventors: Jonathan D. FINN, Melrose, MA (US); Margarita Jacoba Bernadetta Maria VERVOORDELDONK, Maarssen (NL); Paul-Peter TAK, Cambridge (GB)

(21) Appl. No.: 15/517,118

(22) PCT Filed: Oct. 6, 2015
(86) PCT No.: PCT/EP2015/072979

§ 371 Date: Apr. 5, 2017

(30) Foreign Application Priority Data
Oct. 6, 2014 (EP) 14187777.9
Aug. 3, 2015 (EP) 15179501.0

(51) Int. Cl. A61K 48/00; A61K 38/16; A61K 38/00; A61K 31/663; A61K 31/573

(52) U.S. Cl. CPC A61K 48/0083; A61K 48/0075; A61K 48/0008; A61K 31/573; A61K 38/00; A61K 31/663; A61K 38/162

(57) ABSTRACT

The invention relates to the field of andeno-associated virus (AAV) based gene therapy, in particular to the use of a combination of recombinant AAV-transgene vectors with an immunosuppressant and/or empty-AAV capsids. The invention further provides a composition and a kit of parts based on this combination.

### FIELD OF THE INVENTION

The invention relates to the field of adeno-associated virus (AAV) based gene therapy, in particular to the use of a combination of recombinant AAV-transgene vectors with an immunosuppressant and/or empty-AAV capsids. The invention further provides a composition and a kit of parts based on this combination.

### BACKGROUND OF THE INVENTION

Adeno-associated virus (AAV) vectors are the gene transfer vectors of choice since they are considered to have the best safety and efficacy profile for the delivery of genes in humans in vivo. Therefore AAV vectors have been extensively used for in vivo gene therapy and have been shown safe and effective in pre-clinical models as well as in clinical trials. AAV vectors have been successful in phase HI studies for hemophilia B, cystic fibrosis, alpha-1 anti-trypsin deficiency, Parkinson disease, Duchenne muscular dystrophy and Leber's congenital amaurosis (Selot et al., Current Pharmaceutical Biotechnology, 2013, 14, 1072-1082). Alipogene tiparvovec (Glybera®, uniQure) has been granted marketing authorization in Europe as a gene therapy for the treatment of lipoprotein lipase deficiency (LPLD). Despite the promise of AAV based gene therapy approaches for treatment of a variety of disorders, unwanted immune responses (following exposure to wild-type AAV or AAV-based vectors) may limit therapeutic efficacy of AAV vectors. Recently, it was reported that addition of a significant amount of empty capsids to the AAV transgene composition after intravenous administration is able to overcome the inhibitory effect of neutralizing antibodies and has an ameliorating effect on transgene expression in the liver (WO2013/078400; WO2013/123503).

AAV vector based gene therapy has also been applied in rheumatoid arthritis (RA), which is a chronic inflammatory disease that affects ˜1% of the population. The pathology of RA extends throughout the synovial joint. The localized nature of the joint makes in vivo gene therapy very attractive. Therapies providing anti-inflammatory proteins aimed at shifting the balance in RA towards an anti-inflammatory state have been applied. The inventors found that after intra-articular administration of AAV expressing luciferase, not all joints are effectively transduced (usually <50%) and expression in injected joints is quite variable. In order to enable sustained local production of effective doses of therapeutic proteins in the joint, in particular in the rheumatoid synovium, an efficient gene delivery system needs to be developed.

### SUMMARY OF THE INVENTION

The invention provides an rAAV vector composition and an immunosuppressant for use in a treatment comprising gene therapy, wherein the treatment comprises the administration of the rAAV vector composition and the administration of the immunosuppressant to an individual, wherein the rAAV vector composition comprises a rAAV-transgene vector and an empty capsid in a ratio of empty capsid to rAAV-transgene vector of at least 1:1.

In a preferred embodiment, at least one of the rAAV vector composition and immunosuppressant is administered locally.

In a further preferred embodiment, at least one of the rAAV vector composition and the immunosuppressant is administered systemically.

Preferably, the rAAV vector composition is administered locally, preferably at a site comprising a substantial amount of innate immune cells, even more preferably the rAAV vector composition is administered intra-articularly.

In an embodiment, the immunosuppressant is administered locally, preferably at a site comprising a substantial amount of innate immune cells, even more preferably intra-articularly.

In a further embodiment, the immunosuppressant is administered systemically, preferably muscularly or intravenously.

In certain embodiments, the rAAV vector composition and the immunosuppressant are administered sequentially, wherein preferably the immunosuppressant is administered prior to the rAAV vector composition.

In an embodiment, the immunosuppressant is an innate immune cell inhibitor, a cytostatic or purinergic signaling pathway modifying drug such as methotrexate, a non-steroidal anti-inflammatory drug, and/or a immunosuppressant biological such as a macrophage depleting antibody, a TNF blocker, IL-6 blocker and/or an IL-2 blocker. Preferably, the immunosuppressant is an innate immune cell inhibitor, preferably a glucocorticoid and/or a liposomal bisphosphonate.

In a certain embodiments, the transgene comprised in the rAAV-transgene vector encodes a therapeutic protein.

In a preferred embodiment, the gene therapy is for preventing, delaying, curing, reverting and/or treating an inflammatory condition or inflammatory disease, and preferably wherein the transgene encodes a therapeutic anti-inflammatory protein. Preferably, the inflammatory condition or disease is a rheumatic condition or disease. Preferably, the gene therapy is for treating, preventing, delaying, curing, reverting and/or treating a non-inflammatory condition or non-inflammatory disease.

In certain embodiments, the rAAV vector composition further comprises a pharmaceutically acceptable carrier, diluents, solubilizer, filler, preservative and/or excipient.

In certain embodiments, the immunosuppressant is comprised within the rAAV vector composition.

The invention further provides a composition comprising a rAAV-transgene vector as defined herein and an empty capsid as defined herein in a ratio of empty capsid to rAAV-transgene vector of at least 1:1, and an immunosuppressant as defined herein.

The invention also provides a kit of parts comprising a rAAV vector composition as defined herein and an immunosuppressant as defined herein.

### DESCRIPTION OF THE INVENTION

### Definitions

A “rAAV-transgene vector” refers to a recombinant adeno-associated virus (AAV) vector which is derived from the wild type AAV by using molecular methods. A rAAV-transgene vector is distinguished from a wild type (wt) AAV vector, since all or a part of the viral genome has been replaced with a transgene, which is a non-native nucleic acid with respect to the AAV nucleic acid sequence as further defined herein. Wild type AAV belongs to the genus Dependovirus, which in turn belongs to the subfamily of the Parvovirinae, also referred to as parvoviruses, which are capable of infecting vertabrates. Parvovirinae belong to family of small DNA animal viruses, i.e. the Parvoviridae family. As may be deduced from the name of their genus, members of the Dependovirus are unique in that they usually require coinfection with a helper virus such as adenovirus or herpes virus for productive infection in cell culture. The genus Dependovirus includes AAV, which normally infects humans, and related viruses that infect other warm-blooded animals (e.g., bovine, canine, equine, and ovine adeno-associated viruses). Further information on parvoviruses and other members of the Parvoviridae is described in Kenneth I. Berns, “Parvoviridae: The Viruses and Their Replication,” Chapter 69 in Fields Virology (3d Ed. 1996). For convenience the present invention is further exemplified and described herein by reference to AAV. It is however understood that the invention is not limited to AAV but may equally be applied to other parvoviruses.

The genomic organization of all known AAV serotypes is very similar. The genome of AAV is a linear, single-stranded DNA molecule that is less than about 5,000 nucleotides (nt) in length. Inverted terminal repeats (ITRs) flank the unique coding nucleotide sequences for the non-structural replication (Rep) proteins and the structural (VP) proteins. The VP proteins (VP1, -2 and -3) form the capsid or protein shell. The terminal 145 nt are self-complementary and are organized so that an energetically stable intramolecular duplex forming a T-shaped hairpin may be formed. These hairpin structures function as an origin for viral DNA replication, serving as primers for the cellular DNA polymerase complex. Following wtAAV infection in mammalian cells the Rep genes 25 (i.e. Rep78 and Rep52) are expressed from the P5 promoter and the P19 promoter, respectively and both Rep proteins have a function in the replication of the viral genome. A splicing event in the Rep ORF results in the expression of actually four Rep proteins (i.e. Rep78, Rep68, Rep52 and Rep40). However, it has been shown that the unspliced mRNA, encoding Rep78 and Rep52 proteins, in mammalian cells are sufficient for AAV vector production. wtAAV infection in mammalian cells relies for the capsid proteins production on a combination of alternate usage of two splice acceptor sites and the suboptimal utilization of an ACG initiation codon for VP2.

A rAAV-transgene vector may have one or preferably all wild type AAV genes deleted, but may still comprise functional ITR nucleic acid sequences. Preferably, the rAAV-transgene vector does not comprise any nucleotide sequences encoding viral proteins, such as the rep (replication) or cap (capsid) genes of AAV. Functional ITR sequences are necessary for the replication, rescue and packaging of AAV virions. The ITR sequences may be wild type sequences or may have at least 80%, 85%, 90%, 95%, or 100% sequence identity with wild type sequences or may be altered by for example in insertion, mutation, deletion or substitution of nucleotides, as long as they remain functional. In this context, functionality refers to the ability to direct packaging of the genome into the capsid shell and then allow for expression in the host cell to be transduced or target cell. Typically, the inverted terminal repeats of the wild type AAV genome are retained in the rAAV-transgene vector. The ITRs can be cloned from the AAV viral genome or excised from a vector comprising the AAV ITRs. The ITR nucleotide sequences can be either ligated at either end to a transgene as defined herein using standard molecular biology techniques, or the wild type AAV sequence between the ITRs can be replaced with the desired nucleotide sequence. The rAAV-transgene vector preferably comprises at least the nucleotide sequences of the inverted terminal repeat regions (ITR) of one of the AAV serotypes, or nucleotide sequences substantially identical thereto, and at least one nucleotide sequence encoding a therapeutic protein (under control of a suitable regulatory element) inserted between the two ITRs. The majority of currently used rAAV-transgene vectors use the ITR sequences from AAV serotype 2. Preferred ITR sequences are represented by the SEQ ID NO: 1-6 as indicated Table 1. Most preferred ITR present in a rAAV-transgene vector is AAV2 ITR. A rAAV genome can comprise of single stranded or double stranded (self-complementary) DNA. The single stranded nucleic acid molecule is either sense or antisense strand, as both polarities are equally capable of gene expression. Single stranded rAAV-transgene vectors may utilize the wild-type AAV serotype 2 (AAV2) ITR sequences (SEQ ID: 24, 25), and double stranded (self-complementary) rAAV-transgene vectors may utilize a modified version of the ITRs (SEQ ID: 26, 27). The rAAV-transgene vector may further comprise a marker or reporter gene, such as a gene for example encoding an antibiotic resistance gene, a fluorescent protein (e.g., gfp) or a gene encoding a chemically, enzymatically or otherwise detectable and/or selectable product (e.g., lacZ, aph, etc.) known in the art.

The rAAV-transgene vector, including any possible combination of AAV serotype capsid and AAV genome ITRs, is produced using methods known in the art, as described in Pan et al. (J. of Virology (1999) 73: 3410-3417), Clark et al. (Human Gene Therapy (1999) 10: 1031-1039), Wang et al. (Methods Mol. Biol. (2011) 807: 361-404) and Grimm (Methods (2002) 28(2): 146-157), which are incorporated herein by reference. In short, the methods generally involve (a) the introduction of the rAAV genome construct into a host cell, (b) the introduction of an AAV helper construct into the host cell, wherein the helper construct comprises the viral functions missing from the wild type rAAV genome and (c) introducing a helper virus construct into the host cell. All functions for rAAV vector replication and packaging need to be present, to achieve replication and packaging of the rAAV genome into rAAV vectors. The introduction into the host cell can be carried out using standard molecular biology techniques and can be simultaneously or sequentially. Finally, the host cells are cultured to produce rAAV vectors and are purified using standard techniques such as CsCl gradients (Xiao et al. 1996, J. Virol. 70: 8098-8108). The purified rAAV vector is then ready for use in the methods. High titres of more than 10′² particles per ml and high purity (free of detectable helper and wild type viruses) can be achieved (Clark et al. supra and Flotte et al. 1995, Gene Ther. 2: 29-37). The total size of the transgene inserted into the rAAV vector between the ITR regions is generally smaller than 5 kilobases (kb) in size.

The sequence encoding the capsid protein can be a capsid sequence as found in nature such as for example of AAV2, AAV5 and AAV8 of which the nucleotide and amino acid sequences are shown in SEQ ID NO: 7-18. In a preferred embodiment, the AAV capsid proteins are AAV serotype 5 or AAV serotype 8 capsid proteins. Alternatively, the sequence is man-made, for example, the sequence may be a hybrid form or may be codon optimized, such as for example by codon usage of AcmNPv or Spodoptera frugiperda. For example, the capsid sequence may be composed of the VP2 and VP3 sequences of AAV1 whereas the remainder of the VP1 sequence is of AAV5. The man-made sequence may result of rational design or directed evolution experiments. This can include generation of capsid libraries via DNA shuffling, error prone PCR, bioinformatic rational design, site saturated mutagenesis. Resulting capsids are based on the existing serotypes but contain various amino acid or nucleotide changes that improve the features of such capsids. The resulting capsids can be a combination of various parts of existing serotypes, “shuffled capsids” or contain completely novel changes, i.e. additions, deletions or substitutions of one or more amino acids or nucleotides, organized in groups or spread over the whole length of gene or protein. See for example Schaffer and Maheshri; Proceedings of the 26th Annual International Conference of the IEEE EMBS San Francisco, Calif., USA; Sep. 1-5, 2004, pages 3520-3523; Asuri et al. (2012) Molecular Therapy 20(2):329-3389; Lisowski et al. (2014) Nature 506(7488):382-386, herein incorporated by reference.

In the context of the present invention a capsid protein shell may be of a different serotype than the rAAV-transgene vector genome ITR. A rAAV-transgene vector of the invention may thus be encapsidated by a capsid protein shell, i.e. the icosahedral capsid, which comprises capsid proteins (VP1, VP2, and/or VP3) of one AAV serotype, e.g., AAV serotype 5, whereas the ITRs sequences contained in that rAAV-transgene vector may be any of the rAAV serotypes described above, including a rAAV5 vector. Preferred wild type capsid protein shell sequences are represented by the SEQ ID NO: 7-18 as indicated in Table 1. In an embodiment, a rAAV-transgene vector is encapsidated by a capsid protein shell of AAV serotype 5 or AAV serotype 2 or AAV serotype 8 wherein the rAAV genome or ITRs present in said rAAV-transgene vector are derived from AAV serotype 2 or AAV serotype 5 (encoded by SEQ ID NO: 5 and 6) or AAV serotype 8. In this embodiment, it is further preferred that a rAAV-transgene vector is encapsidated by a capsid protein shell of the AAV serotype 5 (more preferably SEQ ID NO: 12, 13, 14 encoded by SEQ ID NO: 11) and the rAAV genome or ITRs present in said rAAV-transgene vector are derived from AAV serotype 2 (more preferably single stranded as SEQ ID NO: 1, 2 or double stranded as SEQ ID NO: 3, 4). This embodiment is preferred for local delivery of a gene to a joint.

In another embodiment it is preferred that a rAAV-transgene vector is encapsidated by a capsid protein shell of the AAV serotype 8 (more preferably SEQ ID NO: 16, 17, 18 encoded by SEQ ID NO: 15) and the rAAV genome or ITRs present in said vector are derived from AAV serotype 2 (more preferably single stranded as SEQ ID NO: 1, 2 or double stranded as SEQ ID NO: 3, 4). This embodiment is preferred for systemic delivery.

In yet another embodiment, it is preferred that a rAAV-transgene vector is encapsidated by a capsid protein shell of the AAV serotype 2 (more preferably SEQ ID NO: 8, 9, 10 encoded by SEQ ID NO: 7) and the rAAV genome or ITRs present in said vector is derived from AAV serotype 2 (more preferably single stranded as SEQ ID NO: 1, 2, or double stranded as SEQ ID NO: 3, 4). The complete genome of AAV5 and other AAV serotypes has been sequenced (Chiorini et al. 1999, J. of Virology Vol. 73, No. 2, p 1309-1319) and the nucleotide sequence is available in GenBank (Accession No. AF085716). The ITR nucleotide sequences of AAV5 are thus readily available to a skilled person. The complete genome of AAV2 is available in NCBI (NCBI Reference Sequence NC_001401.2). They can be either cloned or made by chemical synthesis as known in the art, using for example an oligonucleotide synthesizer as supplied e.g., by Applied Biosystems Inc. (Fosters, Calif., USA) or by standard molecular biology techniques.

A “serotype” is traditionally defined on the basis of a lack of cross-reactivity between antibodies to one virus as compared to another virus. Such cross-reactivity differences are usually due to differences in capsid protein sequences/antigenic determinants (e.g., due to VP1, VP2, and/or VP3 sequence differences of AAV serotypes). Under the traditional definition, a serotype means that the virus of interest has been tested against serum specific for all existing and characterized serotypes for neutralizing activity and no antibodies have been found that neutralize the virus of interest. As more naturally occurring virus isolates are discovered and capsid mutants generated, there may or may not be serological differences with any of the currently existing serotypes. Thus, in cases where the new AAV has no serological difference, this new AAV would be a subgroup or variant of the corresponding serotype. In many cases, serology testing for neutralizing activity has yet to be performed on mutant viruses with capsid sequence modifications to determine if they are of another serotype according to the traditional definition of serotype. Accordingly, for the sake of convenience and to avoid repetition, the term “serotype” broadly refers to both serologically distinct viruses (e.g., AAV) as well as viruses (e.g., AAV) that are not serologically distinct that may be within a subgroup or variant of a given serotype. By way of example, rAAV-transgene vector include various naturally and non-naturally occurring serotypes. Such non-limiting serotypes include AAV-1, -2, -3, -4, -5, -6, -7, -8, -9, -10, -11, -rh74, -rh10, AAV-DJ and AAV-2i8. Again, for the sake of convenience, serotypes include AAV with capsid sequence modifications that have not been fully characterized as being a distinct serotype, and may in fact actually constitute a subgroup or variant of a known serotype.

“Empty-AAV capsid”, also denominated herein as “empty capsid”, is constituted only by a capsid protein and is free from a viral nucleic acid genome. Empty capsids are virus-like particles in that they bind with one or more antibodies or scavenger receptors that bind with the full (genome containing) vector (e.g., adeno-associated virus, AAV) thereby preferably functioning as a decoy to reduce immune responses against the viral vector. Such a decoy preferably acts to absorb the antibodies or scavenger receptors directed against the viral vector, thereby increasing or improving viral vector transgene transduction of cells (introduction of the transgene) in the context of such antibodies or scavenger receptors, and in turn increasing cellular expression of the gene transcript and/or encoded protein. For example, an empty AAV8 capsid would retain the ability to bind with one or more antibodies or scavenger receptors that bind to an AAV, such as a wild type AAV8 or a rAAV8-transgene vector, or another AAV serotype. For example, an empty AAV2 capsid would retain the ability to bind with one or more antibodies or scavenger receptors that bind to wild type AAV8 or a rAAV8-transgene vector. Empty capsids may retain the ability to enter a cell, but are not required to enter a cell, for example, modifying or cross-linking a capsid protein sequence of empty capsids reduces the ability of the modified or cross-linked capsids to enter cells. Thus, such empty capsids may have reduced binding to a cell as compared to a viral vector that includes the transgene. Accordingly, empty capsids may be unmodified, or modified and have reduced binding to a cell as compared to a viral vector that includes the transgene. In particular embodiments, empty capsids are treated with a cross-linking agent, or comprise mutated capsids that exhibit reduced or decreased binding to AAV receptor. In particular aspects, a mutated capsid comprises one or more mutated capsid proteins as disclosed in WO2013/078400, i.e. capsid proteins wherein one or more arginine (R) residues that contribute to heparin sulfate proteoglycan binding has been substituted with a non-charged or hydrophobic residue, or any AAV capsid protein, such as AAV2 VP1 (SEQ ID NO: 8) and/or VP2 (SEQ ID NO: 9) with one or more arginine (R) residues substituted at any of the following positions: 451, 448, 530, 585 or 588 (e.g., one or more arginine (R) residues substituted at any of position: 451 with a cysteine, 448 with a cysteine, 530 with an alanine, 585 with an alanine or 588 with an alanine).

Empty-AAV capsids or empty capsids are sometimes naturally found in AAV vector preparations. Such natural mixtures can be used in accordance with the invention, or if desired be manipulated to increase or decrease the amount of empty capsid and/or vector. Not wished to be bound by any theory, the empty capsids may act as a decoy, thereby prevent degradation of a rAAV-transgene vector. In this case, the amount of empty capsid can be adjusted to an amount that would be expected to reduce the inhibitory effect of antibodies or macrophages that react with or bind to an rAAV vector that is intended to be used for vector-mediated gene transduction in the individual.

Empty capsids can also be produced independent of AAV vector preparations, and if desired, added to AAV vector preparations, or administered separately to an individual. Empty capsids, genome containing capsids and capsid proteins can be generated and purified and their quantities determined, optionally adjusted, for example, according to AAV antibody titer or serotype in the individual, and used or administered according to their intended purpose.

An “innate immune cell” is understood herein as a neutrophil, macrophage, monocyte, eosinophil, basophil, or dendritic cell, that has the potential to participate in the inflammatory response to a foreign substance.

A “macrophage” is understood herein as an innate immune cell that engulfs and digests cellular debris, foreign substances, microbes, and cancer cells in a process called phagocytosis.

The term “transgene” is used to refer to a non-native nucleic acid with respect to the AAV nucleic acid sequence. It is used to refer to a polynucleotide that can be introduced into a cell or organism. Transgenes include any polynucleotide, such as a gene that encodes a polypeptide or protein, a polynucleotide that is transcribed into an inhibitory polynucleotide, or a polynucleotide that is not transcribed (e.g., lacks a expression control element, such as a promoter that drives transcription). A transgene of the invention may comprises at least two nucleotide sequences each being different or encoding for different therapeutic molecules. The at least two different nucleotide sequences may be linked by an IRES (internal ribosome entry sites) element, providing a bicistronic transcript under control of a single promoter. Suitable IRES elements are described in e.g., Hsieh et al. (1995, Biochemical Biophys. Res. Commun. 214:910-917). Furthermore, the at least two different nucleotide sequences encoding for different (therapeutic) polypeptides or proteins may be linked by a viral 2A sequence to allow for efficient expression of both transgenes from a single promoter. Examples of 2A sequences include foot and mouth disease virus, equine rhinitis A virus, Thosea asigna virus and porcine teschovirus-1 (Kim et al., PLoS One (2011) 6(4): e18556). A transgene is preferably inserted within the rAAV genome or between ITR sequences as indicated above. A transgene may also be an expression construct comprising an expression regulatory element such as a promoter or transcription regulatory sequence operably linked to a coding sequence and a 3′ termination sequence. Preferably, the coding sequence within the transgene is not operably linked to a steroid inducible promoter. More preferably, the coding sequence within the transgene is not operably linked to a dexamethasone inducible promoter

In a cell having a transgene, the transgene has been introduced/transferred/transduced by rAAV “transduction” of the cell. A cell or progeny thereof into which the transgene has been introduced is referred to as a “transduced” cell. Typically, a transgene is included in progeny of the transduced cell or becomes a part of the organism that develops from the cell. Accordingly, a “transduced” cell (e.g., in a mammal, such as a cell or tissue or organ cell), means a genetic change in a cell following incorporation of an exogenous molecule, for example, a polynucleotide or protein (e.g., a transgene) into the cell. Thus, a “transduced” cell is a cell into which, or a progeny thereof in which an exogenous molecule has been introduced, for example. The cell(s) can be propagated and the introduced protein expressed, or nucleic acid transcribed.

“Transduction” refers to the transfer of a transgene into a recipient host cell by a viral vector. Transduction of a target cell by a rAAV-transgene vector of the invention leads to transfer of the transgene contained in that vector into the transduced cell. “Host cell” or “target cell” refers to the cell into which the DNA delivery takes place, such as the synoviocytes or synovial cells of an individual. AAV vectors are able to transduce both dividing and non-dividing cells.

“Gene” or “coding sequence” refers to a DNA or RNA region which “encodes” a particular protein. A coding sequence is transcribed (DNA) and translated (RNA) into a polypeptide when placed under the control of an appropriate regulatory region, such as a promoter. A gene may comprise several operably linked fragments, such as a promoter, a 5′ leader sequence, an intron, a coding sequence and a 3′nontranslated sequence, comprising a polyadenylation site or a signal sequence. A chimeric or recombinant gene is a gene not normally found in nature, such as a gene in which for example the promoter is not associated in nature with part or all of the transcribed DNA region. “Expression of a gene” refers to the process wherein a gene is transcribed into an RNA and/or translated into an active protein.

As used herein, the term “promoter” or “transcription regulatory sequence” refers to a nucleic acid fragment that functions to control the transcription of one or more coding sequences, and is located upstream with respect to the direction of transcription of the transcription initiation site of the coding sequence, and is structurally identified by the presence of a binding site for DNA-dependent RNA polymerase, transcription initiation sites and any other DNA sequences, including, but not limited to transcription factor binding sites, repressor and activator protein binding sites, and any other sequences of nucleotides known to one of skill in the art to act directly or indirectly to regulate the amount of transcription from the promoter. A “constitutive” promoter is a promoter that is active in most tissues under most physiological and developmental conditions. An “inducible” promoter is a promoter that is physiologically or developmentally regulated, e.g., by the application of a chemical inducer. A preferred inducible promoter is an NF-Kb responsive promoter which is inducible upon inflammation. A more preferred NF-Kb responsive promoter comprises SEQ ID NO: 19. A “tissue specific” promoter is preferentially active in specific types of tissues or cells. The selection of an appropriate promoter sequence generally depends upon the host cell selected for the expression of a DNA segment. Preferred promoter sequences within the rAAV and/or transgene of the invention are promoters which confer expression in cells of the rheumatoid synovium, such as in intimal macrophages and/or in fibroblast-like synoviocytes and/or other synovial cells such as, but not limited to, T-cells. Preferred promoters are for example the promoters of genes known to be expressed in synovial cells, such as the CMV promoter (cytomegalovirus), the promoter of the IL-6 gene or the SV40 promoter, or an NF-κB inducible promoter as earlier identified herein and others, as readily determined by a skilled person. Alternatively a transgene is be operably linked to a promoter that allows for efficient systemic expression. Suitable promoter sequences are CMV promoter, CBA (chicken beta actin), or liver specific promoters such as human alpha-1 anti-trypsin (hAAT) or TBG (thyroxine binding globulin). Preferably, promoter within the rAAV and/or transgene is not a steroid inducible promoter. More preferably, the promoter within the rAAV and/or transgene is not a dexamethasone inducible promoter.

As used herein, the term “operably linked” refers to a linkage of polynucleotide (or polypeptide) elements in a functional relationship. A nucleic acid is “operably linked” when it is placed into a functional relationship with another nucleic acid sequence. For instance, a transcription regulatory sequence is operably linked to a coding sequence if it affects the transcription of the coding sequence. “Operably linked” means that the DNA sequences being linked are typically contiguous and, where necessary to join two protein encoding regions, contiguous and in reading frame.

As used herein, “gene therapy” is the insertion of nucleic acid sequences (e.g., a transgene as defined herein) into an individual's cells and/or tissues to treat a disease. The transgene can be a functional mutant allele that replaces or supplements a defective one. Gene therapy also includes insertion of transgene that are inhibitory in nature, i.e., that inhibit, decrease or reduce expression, activity or function of an endogenous gene or protein, such as an undesirable or aberrant (e.g., pathogenic) gene or protein. Such transgenes may be exogenous. An exogenous molecule or sequence is understood to be molecule or sequence not normally occurring in the cell, tissue and/or individual to be treated. Both acquired and congenital diseases are amenable to gene therapy.

A “therapeutic polypeptide” or “therapeutic protein” is to be understood herein as a polypeptide or protein that can have a beneficial effect on an individual, preferably said individual is a human, more preferably said human suffers from a disease. Such therapeutic polypeptide may be selected from, but is not limited to, the group consisting of an enzyme, a co-factor, a cytokine, an antibody, a growth factor, a hormone and an anti-inflammatory protein.

A “therapeutically-effective” amount as used herein is an amount that is sufficient to alleviate (e.g., mitigate, decrease, reduce) at least one of the symptoms associated with a disease state. Alternatively stated, a “therapeutically-effective” amount is an amount that is sufficient to provide some improvement in the condition of the individual.

“Sequence identity” is herein defined as a relationship between two or more amino acid (polypeptide or protein) sequences or two or more nucleic acid (polynucleotide) sequences, as determined by comparing the sequences. In a preferred embodiment, sequence identity is calculated based on the full length of two given SEQ ID NO or on part thereof. Part thereof preferably means at least 10%, 20%, 30%, 40%, 50%, 60%, 70%, 80%, 90%, or 100% of both SEQ ID NO. In the art, “identity” also means the degree of sequence relatedness between amino acid or nucleic acid sequences, as the case may be, as determined by the match between strings of such sequences. Unless otherwise indicated herein, identity or similarity with a given SEQ ID NO means identity or similarity based on the full length of said sequence (i.e. over its whole length or as a whole).

“Similarity” between two amino acid sequences is determined by comparing the amino acid sequence and its conserved amino acid substitutes of one polypeptide to the sequence of a second polypeptide. “Identity” and “similarity” can be readily calculated by known methods, including but not limited to those described in (Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heine, G., Academic Press, 1987; and Sequence Analysis Primer, Gribskov, M. and Devereux, J., eds., M Stockton Press, New York, 1991; and Carillo, H., and Lipman, D., SIAM J. Applied Math., 48:1073 (1988).

Preferred methods to determine identity are designed to give the largest match between the sequences tested. Methods to determine identity and similarity are codified in publicly available computer programs. Preferred computer program methods to determine identity and similarity between two sequences include e.g., the GCG program package (Devereux, J., et al., Nucleic Acids Research 12 (1): 387 (1984)), BestFit, BLASTP, BLASTN, and FASTA (Altschul, S. F. et al., J. Mol. Biol. 215:403-410 (1990). The BLAST X program is publicly available from NCBI and other sources (BLAST Manual, Altschul, S. et al., NCBI NLM NIH Bethesda, Md. 20894; Altschul, S. et al., J. Mol. Biol. 215:403-410 (1990). The well-known Smith Waterman algorithm may also be used to determine identity.

Preferred parameters for polypeptide sequence comparison include the following: Algorithm: Needleman and Wunsch, J. Mol. Biol. 48:443-453 (1970); Comparison matrix: BLOSSUM62 from Hentikoff and Hentikoff, Proc. Natl. Acad. Sci. USA. 89:10915-10919 (1992); Gap Penalty: 12; and Gap Length Penalty: 4. A program useful with these parameters is publicly available as the “Ogap” program from Genetics Computer Group, located in Madison, Wis. The aforementioned parameters are the default parameters for amino acid comparisons (along with no penalty for end gaps).

Preferred parameters for nucleic acid comparison include the following: Algorithm: Needleman and Wunsch, J. Mol. Biol. 48:443-453 (1970); Comparison matrix: matches=+10, mismatch=0; Gap Penalty: 50; Gap Length Penalty: 3. Available as the Gap program from Genetics Computer Group, located in Madison, Wis. Given above are the default parameters for nucleic acid comparisons.

Optionally, in determining the degree of amino acid similarity, the skilled person may also take into account so-called “conservative” amino acid substitutions, as will be clear to the skilled person. Conservative amino acid substitutions refer to the interchangeability of residues having similar side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulphur-containing side chains is cysteine and methionine. Preferred conservative amino acids substitution groups are: valine-leucine-isoleucine, phenylalanine-tyrosine, lysine-arginine, alanine-valine, and asparagine-glutamine. Substitutional variants of the amino acid sequence disclosed herein are those in which at least one residue in the disclosed sequences has been removed and a different residue inserted in its place. Preferably, the amino acid change is conservative. Preferred conservative substitutions for each of the naturally occurring amino acids are as follows: Ala to Ser; Arg to Lys; Asn to Gln or His; Asp to Glu; Cys to Ser or Ala; Gln to Asn; Glu to Asp; Gly to Pro; His to Asn or Gln; Ile to Leu or Val; Leu to Ile or Val; Lys to Arg; Gln or Glu; Met to Leu or Ile; Phe to Met, Leu or Tyr; Ser to Thr; Thr to Ser; Trp to Tyr; Tyr to Trp or Phe; and, Val to Ile or Leu.

The “synovium” or “synovial tissue” or “synovial cells” as used herein refers to the cellular lining covering the non-cartilaginous surfaces of the synovial joints, as further described in Tak (2000, Examination of the synovium and synovial fluid. In: Firestein G S, Panyani G S, Wollheim F A editors. Rheumatoid Arthritis. New York: Oxford Univ. Press, Inc. 55-68) and incorporated herein by reference. The synovium consists of the intimal lining layer (or synovial lining layer) and the synovial sublining (subsynovium), which merges with the joint capsule. The intimal lining layer comprises intimal macrophages (or macrophage-like synoviocytes or type A synoviocytes) and fibroblast-like synoviocytes (FLS or type B synoviocytes). “Synovium” may therefore be replaced by or is synonymous with “synovial tissue”. A synovial cell can include any cell present in the synovium including FLS and macrophage-like synoviocyte. A synoviocyte cell may also be a neutrophil, T, B cells and/or connective tissue cells, which may all be present in the synovium.

The term “rheumatoid synovium” or “rheumatoid synovial cells” or “rheumatoid synovial tissue” refers to the inflamed synovium of the joints of an individual suffering from rheumatoid arthritis. The rheumatoid synovium is characterized by intimal lining hyperplasia and by accumulation of FLS, T-cells, plasma cells, macrophages, B-cells, natural killer cells and dendritic cells in the synovial sublining. These accumulated cells are comprised in the definition of rheumatoid synovial cells.

In addition, reference to an element by the indefinite article “a” or “an” does not exclude the possibility that more than one of the element is present, unless the context clearly requires that there be one and only one of the elements. The indefinite article “a” or “an” thus usually means “at least one”.

The word “approximately” or “about” when used in association with a numerical value (approximately 10, about 10) preferably means that the value may be the given value of 10 more or less 10% of the value.

### DETAILED DESCRIPTION OF THE INVENTION

In a first aspect, the invention provides a recombinant adeno-associated viral (rAAV) vector composition. Preferably, said rAAV vector composition is for or suitable for application in gene therapy. The rAAV vector composition of the invention comprises at least a rAAV-transgene vector as defined herein. Preferably, the transgene is therapeutically active.

Preferably, the transgene encodes a therapeutic (poly)peptide or therapeutic protein. Therapeutic (poly)peptides and proteins for use in the context of the present invention include, but are not limited to, (soluble) cluster of differentiation 39 (CD39) protein, (soluble) cluster of differentiation 73 (CD73) protein, Recombinant Anti-Inflammation fusioN protein (RAIN) (CD73-39 fusion), interleukin-1 inhibitor, tumor necrosis factor-α inhibitor, interleukin-12 inhibitor, interleukin-1 receptor antagonist, interleukin-18 binding protein, soluble tumor necrosis factor-α receptor p55 or soluble tumor necrosis factor-α protein 75, dominant negative IκB kinase-β, interleukin-4, interleukin-10, interleukin-13, interferon-β, vasoactive intestinal polypeptide, cystic fibrosis transmembrane regulator protein (CFTR), dystrophin, utrophin, blood coagulation (clotting) factor (e.g., Factor XIII, Factor IX, Factor X, Factor VIII, Factor VIIa, protein C, Factor VII, B domain-deleted Factor VIII, or a high-activity or longer half life variant of coagulation factor, or an active or inactive form of a coagulation factor), a monoclonal antibody (e.g., against tumor necrosis factor-α or interleukin-12), retinal pigment epithelium-specific 65 kDa protein (RPE65), erythropoietin, LDL receptor, lipoprotein lipase, ornithine transcarbamylase, β-globin, α-globin, spectrin, α-antitrypsin, adenosine deaminase (ADA), a metal transporter (ATP7A or ATP7), sulfamidase, an enzyme involved in lysosomal storage disease (ARSA), hypoxanthine guanine phosphoribosyl transferase, P-25 glucocerebrosidase, sphingomyelinase, lysosomal hexosaminidase, branched chain keto acid dehydrogenase, a hormone, a growth factor, insulin-like growth factor 1 or 2, platelet derived growth factor, epidermal growth factor, nerve growth factor, neurotrophic factor-3 and -4, brain-derived neurotrophic factor, glial derived growth factor, transforming growth factor α and β, a cytokine, interferon-α, interferon-γ, interleukin-2, interleukin-12, granulocyte-macrophage colony stimulating factor, lymphotoxin, a suicide gene product, herpes simplex virus thymidine kinase, cytosine deaminase, diphtheria toxin, cytochrome P450, deoxycytidine kinase, tumor necrosis factor, a drug resistance protein, a tumor suppressor protein (e.g., p53, Rb, Wt-1, NF1, Von Hippel-Lindau (VHL), SERCA2a, adenomatous polyposis coli (APC)), VEGF, microdystrophin, lysosomal acid lipase, arylsulfatase A and B, ATP7A and B, a peptide with immunomodulatory properties, a tolerogenic or immunogenic peptide or protein Tregitope or hCDR1, insulin, glucokinase, guanylate cyclase 2D (LCA-GUCY2D), Rab escort protein 1 (Choroideremia), LCA 5 (LCA-Lebercilin), ornithine ketoacid aminotransferase (Gyrate Atrophy), Retinoschisin 1 (X-linked Retinoschisis), USH1C (Usher's Syndrome IC), X-linked retinitis pigmentosa GTPase (XLRP), MERTK (AR forms of RP: retinitis pigmentosa), DFNB1 (Connexin 26 deafness), ACHM 2, 3 and 4 (Achromatopsia), PKD-1 or PKD-2 (Polycystic kidney disease), TPP1, CLN2, a gene product implicated in lysosomal storage diseases (e.g., sulfatases, N-acetylglucosamine-1-phosphate transferase, cathepsin A, GM2-AP, NPC1, VPC2, a sphingolipid activator protein), or one or more zinc finger nucleases, transcription activation-like effector nucleases (TALENs), or CRISPER-Cas9 protein for genome editing, or donor sequences used as repair templates for genome editing, and any other peptide or protein that has a therapeutic effect in an individual in need thereof. Preferably, the therapeutic protein is a therapeutic anti-inflammatory protein, preferably selected from the group consisting of (soluble) cluster of differentiation 39 (CD39) protein, (soluble) cluster of differentiation 73 (CD73) protein, interleukin-1 inhibitor, tumor necrosis factor-α inhibitor, interleukin-1 receptor antagonist, interleukin-18 binding protein, soluble tumor necrosis factor-α receptor p55 or soluble tumor necrosis factor-α protein 75, dominant negative IκB kinase-β, interleukin-4, interleukin-10, interleukin-13, interferon-β and vasoactive intestinal polypeptide.

Further exemplary therapeutic peptides or proteins encoded by transgenes include those that may be used in the treatment of a disease or disorder including, but not limited to, rheumatoid arthritis (RA), juvenile rheumatoid arthritis, osteoarthritis (OA), gout, spondlyarthritis (SpA), psoriasis, psoriatic arthritis, ankylosing spondylitis, inflammatory bowel disease including Crohn's disease or ulcerative colitis, hepatitis, sepsis, alcoholic liver disease, and non-alcoholic steatosis, cystic fibrosis (and other diseases of the lung), hemophilia A, hemophilia B, thalassemia, anemia and other blood disorders, AIDS, Alzheimer's disease, Parkinson's disease, Huntington's disease, amyotrophic lateral sclerosis, epilepsy, and other neurological disorders, cancer, diabetes mellitus, muscular dystrophies (e.g., Duchenne, Becker), Gaucher's disease, Hurler's disease, adenosine deaminase deficiency, glycogen storage diseases and other metabolic defects, retinal degenerative diseases (and other diseases of the eye), and diseases of solid organs (e.g., brain, liver, kidney, heart).

As set forth herein, the transgene of the invention may be an inhibitory and/or antisense nucleic acid sequence Inhibitory, antisense, siRNA, miRNA, shRNA, RNAi and antisense oligonucleotides can modulate expression of a target gene. Such molecules include those able to inhibit expression of a target gene involved in mediation of a disease process, thereby reducing, inhibiting or alleviating one or more symptoms of a disease.

Antisense includes single, double or triple stranded polynucleotides and peptide nucleic acids (PNAs) that bind RNA transcript or DNA (e.g., genomic DNA). Oligonucleotides derived from the transcription initiation site of a target gene, e.g., between positions −10 and +10 from the start site, are another particular example. Triplex forming antisense can bind to double strand DNA thereby inhibiting transcription of the gene. “RNAi” is the use of single or double stranded RNA sequences for inhibiting gene expression (see, e.g., Kennerdell et al., Cell 95:1017 (1998); and Fire et al., Nature, 391:806(1998)). Double stranded RNA sequences from a target gene coding region may therefore be used to inhibit or prevent gene expression/transcription in accordance with the methods and uses of the invention. Antisense and RNAi can be produced based upon nucleic acids encoding target gene sequences (e.g., HTT), such as nucleic acid encoding mammalian and human HTT. For example, a single or double stranded nucleic acid (e.g., RNA) can target HTT transcript (e.g., mRNA).

A “siRNA” refers to a therapeutic molecule involved in the RNA interference process for a sequence-specific post-transcriptional gene silencing or gene knockdown. siRNAs have homology with the sequence of the cognate mRNA of the targeted gene. Small interfering RNAs (siRNAs) can be synthesized in vitro or generated by ribonuclease III cleavage from longer dsRNA and are the mediators of sequence-specific mRNA degradation. siRNA or other such nucleic acids of the invention can be chemically synthesized using appropriately protected ribonucleoside phosphoramidites and a conventional DNA/RNA synthesizer. The siRNA can be synthesized as two separate, complementary RNA molecules, or as a single RNA molecule with two complementary regions. Commercial suppliers of synthetic RNA molecules or synthesis reagents include Applied Biosystems (Foster City, Calif., USA), Proligo (Hamburg, Germany), Dharmacon Research (Lafayette, Colo., USA), Pierce Chemical (part of Perbio Science, Rockford, Ill., USA), Glen Research (Sterling, Va., USA), ChemGenes (Ashland, Mass., USA) and Cruachem (Glasgow, UK). Specific siRNA constructs for inhibiting mRNA of a target gene may be between 15-50 nucleotides in length, and more typically about 20-30 nucleotides in length. Such nucleic acid molecules can be readily incorporated into the viral vectors disclosed herein using conventional methods known to one of skill in the art.

Particular non-limiting examples of genes (e.g., genomic DNA) or transcript of a pathogenic gene (e.g., RNA or mRNA) that may be targeted with inhibitory nucleic acid sequences in accordance with the invention include, but are not limited to: pathogenic genes associated with polynucleotide repeat diseases such as huntingtin (HTT) gene, a gene associated with dentatorubropallidolusyan atropy (e.g., atrophin 1, ATNI); androgen receptor on the X chromosome in spinobulbar muscular atrophy, human Ataxin-1, -2, -3, and −7, Ca, 2. 1 P/Q voltage-dependent calcium channel is encoded by the (CACNA IA), TATA-binding protein, Ataxin 8 opposite strand, also known as ATXN8OS, Serine/threonine-protein phosphatase 2A 55 kDa regulatory subunit B beta isoform in spinocerebellar ataxia (type 1, 2, 3, 6, 7, 8, 12 17), FMR1 (fragile X mental retardation I) in fragile X syndrome, FMR1 (fragile X mental retardation I) in fragile X-associated tremor/ataxia syndrome, FMR1 (fragile X mental retardation 2) or AF4/FMR2 family member 2 in fragile XE mental retardation; Myotonin-protein kinase (MTPK) in myotonic dystrophy; Frataxin in Friedreich's ataxia; a mutant of superoxide dismutase 1 (SOD I) gene in amyotrophic lateral sclerosis; a gene involved in pathogenesis of Parkinson's disease and/or Alzheimer's disease; apolipoprotein B (APOB) and proprotein convertase subtilisin/kexin type 9 (PCSK9), hypercoloesterolemia; HIV Tat, human immunodeficiency virus transactivator of transcription gene, in HIV infection; HIV TAR, HIV TAR, human immunodeficiency virus transactivator response element gene, in HIV infection; C-C chemokine receptor (CCR5) in HIV infection; Rous sarcoma virus (RSV) nucleocapsid protein in RSV infection, liver-specific microRNA (miR-122) in hepatitis C virus infection; p53, acute kidney injury or delayed graft function kidney transplant or kidney injury acute renal failure; protein kinase N3 (PKN3) in advance recurrent or metastatic solid malignancies; LMP2, LMP2 also known as proteasome subunit beta-type 9 (PSMB 9), metastatic melanoma; LMP7, also known as proteasome subunit beta-type 8 (PSMB 8), metastatic melanoma; MECL1 also known as proteasome subunit beta-type 10 (PSMB 10), metastatic melanoma; vascular endothelial growth factor (VEGF) in solid tumors; kinesin spindle protein in solid tumors, apoptosis suppressor Bcell CLL/lymphoma (BCL-2) in chronic myeloid leukemia; ribonucleotide reductase M2 (RRM2) in solid tumors; Furin in solid tumors; polo-like kinase 1 (PLKI) in liver tumors, diacylglycerol 18 acyltransferase 1 (DGATI) in hepatitis C infection, beta-catenin in familial adenomatous polyposis; beta2 adrenergic receptor, glaucoma; RTP801/Reddl also known as DAN damage inducible transcript 4 protein, in diabetic macular edema (DME) or age-related macular degeneration; vascular endothelial growth factor receptor I (VEGFR1) in age-related macular degeneration or choroidal neivascularization, caspase 2 in non-arteritic ischaemic optic neuropathy; Keratin 6A N17K mutant protein in pachyonychia congenital; influenza A virus genome/gene sequences in influenza infection; severe acute respiratory syndrome (SARS) coronavirus genome/gene sequences in SARS infection; respiratory syncytial virus genome/gene sequences in respiratory syncytial virus infection; Ebola filovirus genome/gene sequence in Ebola infection; hepatitis B and C virus genome/gene sequences in hepatitis B and C infection; herpes simplex virus (HSV) genome/gene sequences in HSV infection, coxsackievirus B3 genome/gene sequences in coxsackievirus B3 infection; silencing of a pathogenic allele of a gene (allelespecific silencing) like torsin A (TORIA) in primary dystonia, pan-class I and HLA-allele specific in transplant; pro-inflammatory molecules such as IL-6, IL-1B, TNF, or CCL2 in inflammatory disease; or mutant rhodopsin gene (RHO) in autosomal dominantly inherited retinitis pigmentosa (adRP).

Preferably, the rAAV vector composition comprises the rAAV-transgene vector as defined above and an empty capsid as defined herein. The empty capsid can be of the same serotype or of a different serotype as compared to the rAAV-transgene vector of the composition of the invention. Preferably, the empty capsid is of the same serotype as the rAAV-transgene vector. Preferably, within the rAAV vector composition of the invention, the empty capsid is of the same serotype as the capsid of the rAAV-transgene vector, preferably being either AAV2 or AAV5. However, also encompassed by the invention is a rAAV vector composition wherein the empty capsids have a different serotype as compared to the capsids of the rAAV-transgene vector (such as, but not limited to, AAV2 empty capsids in combination with rAAV-transgene vectors having capsids of the AAV5 serotype, or the other way around). Further encompassed is a rAAV vector composition wherein the empty capsids have a mixture of serotypes, such as, but not limited to, a mixture of AAV2 and AAV5 capsids. The inventors report an increasing effect of transgene expression in joints after intra-articular administration of rAAV-transgene vectors admixed with a significant amount of empty capsids. Preferably in the rAAV-transgene vector and the empty capsid are present within the composition in a ratio of empty capsid to rAAV-transgene vector of at least 1:1, 2:1, 3:1, 4:1, 5:1, 10:1, 15:1, 20:1, 50:1, 100:1, or 1000:1, preferably at least 5:1 (i.e. an amount of empty capsids that is at least 5 times the amount of rAAV-trangene vectors). Preferably said composition comprises rAAV-transgene vector and empty capsids in a ratio of empty capsid to rAAV-transgene vector of at most 10000:1, 5000:1, 4000:1, 3000:1, 2000:1, 1000:1, 500:1, 400:1, 300:1, 200:1, 100:1, 90:1, 80:1, 70:1, 60:1, 50:1, 40:1, 30:1, 20:1, 15:1, 10:1 or 5:1, preferably at most 1000:1. Preferably said composition comprises rAAV-transgene vectors and empty capsids in a ratio of empty capsid to rAAV-transgene vector of between 1:1 to 100:1, 2:1 to 100:1, 5:1 to 100:1, 1:1 to 20:1, 2:1 to 20:1 or preferably between 5:1 to 20:1.

Provided herein above is an embodiment wherein the rAAV-transgene vector and the empty capsids are present in a single composition. Also encompassed within the present invention is an alternative embodiment wherein the rAAV-transgene vector and the empty capsids are present in (at least two or more) separate, distinct compositions. In this alternative embodiment, the rAAV-transgene vector and the empty capsids can be administered separately in time (e.g., sequentially) and/or localization, wherein localization is to be understood as the site of administration. Furthermore, the rAAV-transgene vector and the empty capsids can be administered simultaneously, e.g., at substantially the same timing, optionally at a separate location. All further restrictions with respect to the transgene and the empty capsid ratio to rAAV-transgene vector as indicated for the previous embodiment is repeated for this embodiment.

Preferably, the rAAV vector composition as defined above is used in combination with an immunosuppressant. The inventors surprisingly found an increasing effect of an immunosuppressant on AAV transgene expression when subjects were treated with both immunosuppressants and rAAV-transgenes. This effect is surprising as in the art the effect of glucocorticoids have been tested on AAV gene expression, however, results were rather disappointing. Pfeifer et al. reported that glucocorticoid (dexamethasone) did not have any significant effect on AAV9 gene expression in the lung (Pfeifer et al., Gene Therapy (2011) 18, 1034-1042). Monahan et al. reported no increase in AAV liver gene expression in mice and a small, insignificant increase in gene expression in one dog (Monahan et al., Molecular Therapy (2010) 18, 1907-1916). Furthermore, the inventors discovered a surprising synergistic effect of the immunosuppressant together with empty vectors on rAAV transgene expression. In one embodiment the immunosuppressant is applied separately from the rAAV vector composition, separate meaning separate in location and/or time. In such an embodiment, the immunosuppressant and the rAAV vector composition may be present in separate and distinct compositions. The immunosuppressant, the rAAV-transgene vector and the empty vectors may even each be present each in a separate, distinct composition. In another embodiment, the immunosuppressant and the rAAV vector composition may be present in a single composition. In a further embodiment, the rAAV-transgene vector and the immunosuppressant are present in a single composition, and preferably this composition is used in treatment together with a separate composition comprising the empty capsid. In an even further embodiment, the immunosuppressant and the empty capsid are present in a single composition, and preferably this composition is used in treatment together with a separate composition comprising the rAAV-transgene vector. Therefore, the invention also provides for a composition comprising an empty capsid and an immunosuppressant as defined herein, for a composition comprising a rAAV-transgene vector and an immunosuppressant as defined herein, and for a composition comprising a rAAV vector composition and an immunosuppressant as defined herein.

Preferably, the immunosuppressant of the invention is an innate immune cell inhibitor, preferably a macrophage inhibitor. An innate immune cell is defined herein as an agent that results in a decrease in innate immune cell activity and/or innate immune cell number. A macrophage inhibitor is defined herein as an agent that results in a decrease in macrophage activity and/or macrophage number. Preferably, the innate immune cell or macrophage inhibitor of the invention, results in a decrease of at least 1%, 2%, 5%, 10%, 15%, 20%, 25%, 30%, 35%, 45%, 55%, 65%, 75%, 85%, 95% or preferably of 100% of the number or activity of innate immune cells or macrophages as compared to the initial number or activity of innate immune cells or macrophages before treatment. Innate immune cell or macrophage activity and/or number can be detected by any suitable assay known by the person skilled in the art, such as, but not limited to MTT (3-(4,5-dimethylthiazol-2-yl)2,5-diphenyl tetrazolium bromide) colorimetric assay for testing macrophage cytotoxic activity in vitro as described by Ferrari et al. (Journal of Immunological Methods, 131 (1990) 165-172), by measurement of cytokine levels (e.g., CCL2, TNF), by histological and histochemical detection methods for instance by CD68 labeling or by in vivo magnetic resonance imaging (MRI) detection of superparamagnetic iron oxide (SPIO) uptake by macrophages, preferably after intravenously administration of superparamagnetic iron oxide (SPIO) as reviewed by Yi-Xiang J. Wang (Quant. Imaging Med Surg (2011)1:35-40). The detection can either be in vitro or in vivo. Preferably, in vivo detection is in an animal model, preferably a rat or murine model.

Preferably, the immunosuppressant is a glucocorticoid and/or a bisphosphonate, preferably a liposomal bisphosphonate. Particular non-limiting examples of glucocortocoids are cortisol, cortisone, prednisone, prednisolone, methylprednisolone, dexamethasone, betamethasone, triamcinolone, beclometasone, fludrocortisone acetate, deoxycorticosterone acetate and aldosteron. Preferably, the immunosuppressant is triamcinolone. Particular non-limiting examples of bisphosphonates are etidronate, clodronte, tiludronate, pamidronate, neridronate, olpadronate, alendronate, ibandronate, risedronate and zoledronate. Preferably, the bisphosphonate is a liposome-encapsulated bisphosphonate or liposomal bisphosphonate, preferably liposomal clodronate. Preferably, the glucocorticoid is not dexamethosone. It is to be understood that the inflammatory or macrophage inhibitor of the invention is not limited to a glucocoritcoids and/or a bisphosphonate. For instance, the inflammatory or macrophage inhibitor of the invention can also be a inflammatory or macrophage depleting antibody such as an anti-F4/80 antibody. Preferably, such antibody is a human or humanized antibody. Further relevant immunosuppressants to be used in the present invention are cytostatic drugs (e.g. alkylating agents and/or antimetabolites such as methotrexate), drugs that modify the purinergic signaling pathway (e.g. methotrexate, adensoine analogs, adenosine receptor antagonists or agonists), non-steroidal anti-inflammatory drugs (NSAIDS, e.g. ibuprofen, diclofenac, meloxicam, naproxen, acetylsalicylic acid), biologicals such as TNF blockers (e.g. infliximab, etanercept, adalimumab, certolizumab, golimumab), IL-6 blockers (e.g. tocilizumab), IL-2 blockers (e.g. basiliximab, daclizumab), IL-1β blockers (e.g. anakinra, rilonacept, canakinumab) muromonab, abatacept, and/or rituximab, and/or other compounds such hydroxychloroquine, chloroquine, leflunomide, sulfasalazine, azathioprine, cyclophosphamide, cyclosporine, gold salt and penicillamine.

Preferably, the rAAV vector composition and/or composition comprising empty capsids and/or the composition comprising the immunosuppressant further comprises a pharmaceutically acceptable carrier, diluents, solubilizer, filler, preservative and/or excipient. Such pharmaceutically acceptable carrier, diluents, solubilizer, filler, preservative and/or excipient may for instance be found in Remington: The Science and Practice of Pharmacy, 20th Edition. Baltimore, Md.: Lippincott Williams & Wilkins, 2000.

In a second aspect, the invention provides for a rAAV vector composition according to the first aspect for use in a treatment comprising gene therapy. Furthermore, the invention provides for the use of a rAAV vector composition according to the first aspect for the preparation of a medicament for gene therapy. Also, the invention provides for a method of treatment comprising gene therapy, wherein the method comprises the administration of the rAAV vector composition according to the first aspect.

Preferably, said gene therapy further comprises the administration of an immunosuppressant as defined herein, either present within the rAAV vector composition, or comprised within a separate, distinct composition, i.e. separate and distinct from the rAAV vector composition. At administration, the rAAV vector composition and/or empty capsids and/or immunosuppressant of the invention is delivered to an individual, a cell, tissue or organ of said individual, preferably an individual suffering from a condition or disease as defined herein. Preferably, the rAAV vector composition and the immunosuppressant are administered simultaneously. Simultaneous administration is to be understood herein as administration at more or less the same time, preferably no longer separated in time than 15 min, 30 min, 1 hour, 2 hour, 3 hours, 12 hours or 24 hours, preferably no longer separated in time than 15 min. In another embodiment, the rAAV vector composition and the immunosuppressant are administered sequentially, wherein preferably the immunosuppressant is administered prior to the rAAV vector composition. Preferably, the immunosuppressant is administered at least 1 hour, 3 hours, 12 hours, 24 hours, 2 days, 4 days or 1 week before administration of the the rAAV vector composition. In case the rAAV-transgene vectors and the empty capsids are present in separate compositions, the immunosuppressant may be administered simultaneously or within at least 15 min, 1 hour, 2 hours, 3 hours, 1 day, 2 days or 1 week prior to the empty capsids and the empty capsids in turn are administered simultaneously or within at least 15 min, 1 hour, 2 hours, 3 hours, 1 day, 2 days or 3 days prior to the rAAV-transgene vectors.

Within the embodiments defined herein, the immunosuppressant may be administrated repeatedly, i.e. prior to and/or simultaneously with the rAAV vector composition. As indicated herein above, preferably the rAAV vector composition comprises a significant amount of empty capsids. Furthermore, the invention encompasses the administration of both rAAV-transgene vectors and empty capsids in separate, distinct compositions, which may be administered simultaneously or sequentially in a method or use of the invention. If comprised in separate compositions, the rAAV-transgene vectors and empty capsids are preferably administered simultaneously. In a further embodiment, the empty capsids are administered at most 3 days, 2 days, 1 day, 24 hours, 12 hours, 3 hours, 2 hours, 1 hour, 30 min, 15 min or 5 min, preferably at most 24 hours, prior to rAAV-transgene vector administration. Furthermore, if comprised in separate compositions, the rAAV-transgene vectors and empty capsids are preferably administered at the same site.

A rAAV vector composition and/or empty capsids and/or an immunosuppressant of the invention may be directly or indirectly administrated using suitable means known in the art. Methods and uses of the invention include delivery and administration of the rAAV vector composition and/or empty vector and/or immunosuppressant systemically, regionally or locally, or by any route, for example, by injection, infusion, orally (e.g., ingestion or inhalation), or topically (e.g., transdermally). Exemplary administration and delivery routes include intravenous (i.v.), intra-articular, intraperitoneal (i.p.), intra-arterial, intramuscular, parenteral, subcutaneous, intra-pleural, topical, dermal, intradermal, transdermal, parenterally, e.g. transmucosal, intra-cranial, intra-spinal, oral (alimentary), mucosal, respiration, intranasal, intubation, intrapulmonary, intrapulmonary instillation, buccal, sublingual, intravascular, intrathecal, intracavity, iontophoretic, intraocular, ophthalmic, optical, intraglandular, intraorgan, intralymphatic. Improvements in means for providing an individual or a cell, tissue, organ of said individual with a rAAV vector composition and/or empty capsids and/or an immunosuppressant of the invention, are anticipated considering the progress that has already thus far been achieved. Such future improvements may of course be incorporated to achieve the mentioned effect of the invention. When administering a rAAV vector composition and/or empty capsids and/or an immunosuppressant of the invention, it is preferred that such combination and/or composition is dissolved in a solution that is compatible with the delivery method. For intravenous, subcutaneous, intramuscular, intrathecal, intraarticular and/or intraventricular administration it is preferred that the solution is a physiological salt solution.

Preferably, the rAAV vector composition is administered locally, preferably at a site of the body comprising substantive infiltration of innate immune cells or where a substantive amount of innate immune cells are present, wherein preferably said innate immune cells are monocytes and/or macrophages, even more preferably said innate immune cells are macrophages. Innate immune cell or macrophage infiltration or the presence of a substantive amount of innate immune cells or macrophages can be assessed by methods known by the person skilled in the art, such as by histological and histochemical methods for instance by CD68 labeling or by detecting MRI imaging of macrophage SPIO uptake after intravenous administration as indicated above and/or methods for detection of cytokines such as IL-6, TNF and/or CCL2. Preferably, substantial innate immune cell or macrophage infiltration at a particular site in the body is preferably understood herein as the presence of a number and/or activity of innate immune cells or macrophages of at least 2, 3, 4, 5, 6, 7, 8, 9, 10, 15 or 20 fold, preferably at least 2 fold in comparison to the number and/or activity of innate immune cells or macrophages of a similar site at the detection limit of methods for assessing innate immune cell or macrophage infiltration as defined above. Preferably, substantial innate immune cell or macrophage infiltration at a particular site in the body is preferably understood herein as the presence of a number and/or activity of innate immune cell or macrophages of at least 2, 3, 4, 5, 6, 7, 8, 9, 10, 15 or 20 fold, preferably at least 2 fold in comparison to the number of innate immune cell or macrophages of a similar site after treatment with a therapeutic effective dosage of triamcinolone, preferably as using an innate immune cell or macrophage infiltration assessing method as indicated above. Preferably said therapeutic effective dosage is a dose as known by the skilled person, e.g. 8-16 mg/day orally, 3-48 mg/day intramuscular, 5-40 mg per intra-articular, depending on size of joint. The maximum weekly dose of triamcinolone is 75 mg. Particular non-limiting examples of sites comprising substantial innate immune cell or macrophage infiltration or a substantive amount of innate immune cells or macrophages are joints (intra-articular), sites of inflammation, arthritic joints, sites of injury, atherosclerotic plaques, tumors, in particular invasive tumors, CNS (central nervous system and/or brain), lung, skin, eye, intestine, liver, spleen and adipose tissues. Preferably, a tissue or site comprising a substantive amount of innate immune cells is understood herein as a tissue or site where innate immune cells, preferably macrophages, make up at least 2%, or preferably at least 5%, of the total amount of cells of said tissue or site.

In case an immunosuppressant is present within the rAAV vector composition of the invention, the immunosuppressant is administered at the same site as the rAAV vector composition, i.e. preferably locally as indicated above. In the embodiment wherein the immunosuppressant is comprised within a separate composition distinct from the rAAV vector composition, the immunosuppressant may be administered systemically, preferably intramuscularly or intravenously. The rAAV vector composition may also be administered locally, preferably at a site of the body comprising substantive numbers of macrophages as defined herein, and the immunosuppressant is administered systemically, preferably intramuscularly or intravenously. Also encompassed in the invention is an embodiment wherein the immunosuppressant and the rAAV vector composition, even though present in distinct compositions, are administered at the same site, preferably locally, more preferably intra-articularly. As further indicated herein, administration of such distinct compositions may be either simultaneously or sequentially.

In a preferred embodiment, the therapy of the present invention is for preventing, delaying, curing, reverting and/or treating an inflammatory condition or inflammatory disease. An inflammatory condition or disease may be any condition or disease wherein inflammation can be detected. Inflammation may be detected by the assessment of the concentration of a C-reactive protein and/or of an inflammatory cytokine/chemokine as IL-6, IL-8 or CCL2 in a sample from an individual. The assessment of the concentration of a C-reactive protein and/or of an inflammatory cytokine/chemokine as IL-6, IL-8 or CCL2 may be carried out at the protein level using an ELISA or Western Blotting. The assessment of the concentration of a C-reactive protein and/or of an inflammatory cytokine/chemokine as IL-6, IL-8 or CCL2 may be carried out at the nucleic acid level using PCR. All these assays are known to the skilled person. Assays for the assessment of the presence of an inflammatory cytokine/chemokine as IL-6, IL-8 or CCL2 have been described in the experimental part. A detectable C-reactive protein and/or of an inflammatory cytokine/chemokine as IL-6, IL-8 or CCL2 may be present as a first or early parameter of such an inflammatory disease or condition. A detectable C-reactive protein and/or of an inflammatory cytokine/chemokine as IL-6, IL-8 or CCL2 may be present later on during the course of said inflammatory disease or condition.

An inflammatory disease or condition may be defined as any disease or condition wherein an increased level of ATP and/or an increased level of AMP and/or a decreased (or a reduction of the) ATPase activity level could be assessed in a sample or in a tissue from an individual. An inflammatory disease or condition may be defined as any disease or condition wherein an increased level of adenosine is expected to alleviate a parameter or symptom associated with such inflammatory disease or condition. The increase or decrease as identified in the previous sentence is preferably assessed as explained herein. Particular non-limiting examples of an inflammatory condition, disease or disorder are rheumatoid arthritis (RA), juvenile rheumatoid arthritis, osteoarthritis (OA), gout, spondlyarthritis (SpA), psoriasis, psoriatic arthritis, ankylosing spondylitis, inflammatory bowel disease including Crohn's disease or ulcerative colitis, hepatitis, sepsis, alcoholic liver disease, and non-alcoholic steatosis. An inflammatory condition or disease may further be selected from, but is not limited to, pain, ischemic disorder, glaucoma, asthma, arthritis, cancer, neurodegenerative disorders, chronic disorders, acute inflammation, blood clotting disorders, heart failure, disorder of platelet function and other disorders where inflammation could be detected by a method known by the skilled person (Libby, Arteriscler Thromb Vasc Biol (2012) 32, 20145-20151; Bending et al., Int Immunol (2012) 6: 339-346; Calle and Fernandez, Diabetes Metab (2012) 3:183-191), preferably, further selected from but not limited to, pain, ischemic disorder, glaucoma, arthritis, cancer, neurodegenerative disorders, chronic disorders, acute inflammation, blood clotting disorders, heart failure, disorder of platelet function and other disorders where inflammation could be detected. It is noted that, even though there is currently debate whether osteoarthritis (OA) is to be considered an inflammatory or non-inflammatory disorder, osteoarthritis to be considered as a condition to be prevented, delayed, cured, reverted and/or treated by a method of the present invention.

In the case of Rheumatoid Arthritis (RA), and other types of arthritis (OA, psoriatic arthritis, spondyloarthritis (SpA), gout), inflammation is supposed to occur in a joint and/or in a cartilage and/or in a synovial tissue and/or in a synovial cell and/or in fibroblast-like synoviocyte cell. Each of these tissues and/or cell types is involved, contributes and/or is associated with inflammation. It is therefore encompassed for RA and other types of arthritis (OA, psoriatic arthritis, SpA, gout), that the rAAV-transgene vector of the invention is delivered to a joint and/or in a cartilage and/or in a synovial tissue and/or in a synovial cell and/or in fibroblast-like synoviocyte cell. Preferably said joint, cartilage, synovial tissue and/or synovial cell and/or in fibroblast-like synoviocyte cell are of an individual suffering from the inflammatory disorder. In a preferred embodiment, the administration of a rAAV vector composition of the invention is local or systemic, preferably targeted to any of the types of cells identified above. More preferably the administration is intra-articular. The term “intra-articular” refers to the interior of a joint, e.g., knee, elbow, shoulder, ankle, wrist, etc. Thus, an intra-articular injection is an injection into the space between the bones of a joint. In the knee, “intra-articular” refers to the space between the femur and the tibia, behind and surrounding the patella.

For IBD and Crohn's disease, inflammation primarily occurs in the stomach and intestine (gut). It is therefore encompassed for IBD and Crohn's disease that the rAAV vector composition of the invention is able to be delivered to the stomach and/or the intestine. Preferably said stomach and/or intestine are of an individual suffering from such inflammatory disorder. In a preferred embodiment, the administration of the rAAV vector composition is local or systemic. More preferably the administration is local or systemic and targeted to the stomach and/or the intestine.

For hepatitis and liver disease, inflammation primarily occurs in the liver. It is therefore encompassed for hepatitis and liver diseases that the rAAV-transgene vector of the invention is able to be delivered to the liver. Preferably said liver is of an individual suffering from such inflammatory disorder. In a preferred embodiment, the administration of the rAAV vector composition of the invention is local or systemic. More preferably the administration is local or systemic and targeted to the liver.

For sepsis, inflammation may be systemic. It is therefore encompassed that for such disease the administration of the rAAV vector composition of the invention is systemic, preferably targeting the liver of such patients.

The rAAV-transgene vector dose to achieve a therapeutic effect, e.g., the dose in rAAV-transgene vector genomes/per kilogram of body weight (vg/kg), or transducing units will vary based on several factors including, but not limited to: route of administration, the level of transgene expression required to achieve a therapeutic effect, the specific disease treated, any host immune response to rAAV-transgene vector, a host immune response to the transgene or expression product (protein), and the stability of the protein expressed. One skilled in the art can readily determine a rAAV-transgene vector dose range to treat a patient having a particular disease or disorder based on the aforementioned factors, as well as other factors. Generally, doses will range from at least 1×10⁶, 1×10⁷, 1×10⁸, or more, for example, 1×10⁹, 1×10¹⁰, 1×10¹¹, 1×10¹², 1×10¹³ or 1×10¹⁴, 1×10¹⁵, 1×10¹⁶, or more, vector genomes per kilogram (vg/kg) of the weight of the individual, to achieve a therapeutic effect.

The immunosuppressant dose depends on the type of immunosuppressant. Effective dosages are known by the skilled person. A preferred therapeutic effective dosage of triamcinolone is indicate above. A preferred therapeutic effective dosage of liposomal clodronate is preferably a therapeutic effective dose as known by the skilled person, e.g. preferably 80-320 mg/dose intra-articular, more preferably 160 mg/dose intra-articular (Barrera et al. 2000, Arthritis & Rheumatism Vol 43(9), p1951-1959).

In a more preferred embodiment, the rAAV composition of the invention and immunosuppressant of the invention, either as combination of separate, distinct compositions or as comprised in a single composition, are able to alleviate one or more symptom(s) from a treated patient and/or one or more characteristic(s) or parameter(s) of a cell or tissue from a treated patient is/are improved using a combination or composition of the invention. For instance, for each inflammatory disease, the skilled person knows at least one symptom, parameter or characteristic, values of said parameter or characteristic associated with said disease and how to assess each of them. Below, we give a parameter specific for Rheumatoid arthritis. Rheumatoid arthritis is a disease that is preferably diagnosed after having assessed the index of Disease Activity Score (DAS) or the related DAS28 (van Riel, Best Practice & Research Clinical Rheumatology (2001) 15: 67-76) including the measurements of several parameters and symptoms on an individual. The assessment of said indexes may be carried out by a clinician examining an individual. In a more preferred embodiment, the combination or composition of the invention is able to alleviate one or more symptom(s) from a treated patient and/or one or more characteristic(s) or parameter(s) of a cell or tissue from a treated patient is/are improved using the combination or the composition of the invention when the combination or composition of the invention is able to induce a significant change in DAS or DAS28. Other ways of assessing rheumatoid arthritis are also described (van Riel, Best Practice & Research Clinical Rheumatology (2001) 15: 67-76; and Gester A. M. et al. Baillière's Clinical Immunology (1999) 13: 629-644). A medicament comprising the combination or composition of the invention is able to improve one parameter if after at least one week, one month, six month, one year or more of treatment using a combination and/or a composition of the invention, the value of said parameter has been improved of at least 1%, 2%, 5%, 10% or more by comparison of the value of said parameter before the onset of the treatment. A medicament comprising the combination or composition of the invention is able to alleviate one symptom or one characteristic of a patient or of a cell, tissue or organ or said patient if after at least one week, one month, six month, one year or more of treatment using a combination and/or a composition of the invention, said symptom or characteristic is no longer detectable.

The invention is useful in both human and veterinary medical applications. Suitable individuals include mammals, such as humans. The term “mammal” as used herein includes, but is not limited to, humans, bovines, ovines, caprines, equines, felines, canines, lagomorphs, etc. Human individuals are the most preferred. Human individuals include fetal, neonatal, infant, juvenile and adult individuals. Most preferred are human individuals suffering from any kind or disease or condition as indicated herein.

In a third aspect, the invention provides a kit of parts comprising a rAAV vector composition according the first aspect and an immunosuppressant as defined in the first aspect. Preferably, the kit of parts further comprises instructions for a dosage regime for the rAAV vector composition and the immunosuppressant. These instructions preferably indicate the use of the dosage form to achieve a desirable effect and the amount of dosage form to be taken over a specified time period, preferably as specified in the second aspect herein. The formulations may conveniently be presented in unit dosage form by methods known to those skilled in the art. Preferably, the rAAV vector composition and immunosuppressant are packaged each in a separate unit (or multiples thereof) in an amount that corresponds to the relevant dosage regime for a single administration (or multiples thereof). The package may be in any suitable form, for example a vial, ampoule or cartridge for an injection pen. Preferably, said kit of parts if for use in a treatment comprising gene therapy as defined herein.

All patent and literature references cited in the present specification are hereby incorporated by reference in their entirety. Each embodiment as identified herein may be combined together unless otherwise indicated.

TABLE 1
list of most sequences identified in the application

| Name of the sequence | SEQ ID NO |
|---|---|
| Single stranded AAV2 ITR 5′ | 1 |
| Single stranded AAV2 ITR 3′ | 2 |
| Double stranded AAV2 ITR 5′ | 3 |
| Double stranded AAV2 ITR 3′ | 4 |
| AAV5 ITR 5′ | 5 |
| AAV5 ITR 3′ | 6 |
| AAV2 Capsid DNA | 7 |
| AAV2 Capsid VP1 | 8 |
| AAV2 Capsid VP2 | 9 |
| AAV2 Capsid VP3 | 10 |
| AAV5 Capsid DNA | 11 |
| AAV5 Capsid VP1 | 12 |
| AAV5 Capsid VP2 | 13 |
| AAV5 Capsid VP3 | 14 |
| AAV8 Capsid DNA | 15 |
| AAV8 Capsid VP1 | 16 |
| AAV8 Capsid VP2 | 17 |
| AAV8 Capsid VP3 | 18 |
| NF-Kb responsive promoter | 19 |

### BRIEF DESCRIPTION OF THE DRAWINGS

FIG. 1: Luciferase expression is influenced by vector administration before or after the onset of arthritis. After induction of arthritis, mice (n=5 per group) were injected with 1.5e10 vg of a rAAV5-transgene vector encoding the gene for luciferase (rAAV.CMV.Fluc5) intra-articularly either on day 17 before onset of arthritis or day 24 after onset of arthritis. Imaging was performed 3 days after vector injection and thereafter weekly up to 4 weeks (group day 24) or 5 weeks (group day 17). Inset: Percentage of knee joints expressing a positive signal. A positive signal was defined as a value 1.5 times above the upper limit of the value of control knee joints for at least one measurement in time. Luminescence is shown per group as average; error bars, SEM.

FIG. 2: Effect of addition of liposomal clodronate, triamcinolone and empty capsids on rAAV5-luciferase expression. After induction of arthritis, mice (n=5 per group) were injected with 1.5e10 vg of rAAV5.CMV.FLuc vector intra-articularly. Imaging was performed 3 days after vector injection and thereafter weekly up to 4 weeks. (a) Addition of liposomal clodronate (5 μl/g i.v.) and triamcinolone (5 mg/kg i.m.) resulted in higher levels of luminescence. (b) Addition of empty AAV5 capsids in a 5:1 ratio (empty to full) with genome containing capsids improved luciferase expression. (c) The percentage of knee joints expressing a positive signal was improved 4 to 9 fold. (d) The clinical score showed a tendency to a decrease in triamcinolone treated animals and a tendency to an initial decrease in liposomal clodronate treated animals.

FIG. 3: Improvement of intra-articular rAAV5-luciferase expression by addition of empty capsids and/or triamcinolone. In a CIA model, mice (n=13 per group) were injected with 1.5e10 of rAAV5.CMV.Fluc vector (intra-articular) with or without the addition of empty AAV5 capsid (5:1 empty to full) or triamcinolone (5 mg/kg i.m.). Mice were followed weekly for 1 month and thereafter monthly up till 6 months. (a) Arthritis activity was scored at each time point and a clinical score was calculated. Initially arthritis activity was lower in groups treated with triamcinolone. (b) Luminescence increased up till 4 months for all groups, and then remained stable. (c) Generalized estimating equations analysis showed a significant improvement in luminescence when both triamcinolone and empty capsids were added. The clinical score and luminescence are shown per group as averages; error bars, SEM.

FIG. 4: Comparison of local v.s. systemic triamcinolone administration. In a CIA model (n=18), triamcinolone (5 mg/kg) (or saline) was administered locally (i.a.) or systemically (i.m.), 2 days prior to i.a. administration of rAAV5.CMV.Fluc vector (1.5e10 vg)+empty AAV5 capsid (5:1 empty ratio:full) (intra-articular). Luciferase expression was followed over time.

FIG. 5: Effect of triamcinolone on spleen size and cell populations in different tissues. Arthritis was induced in several groups (n=5 per group), triamcinolone (5 mg/kg) or saline as control was administered intramuscularly on day 22 (2 days before vector administration). Tissues were analysed by FLOW cytometry 48 hrs after triamcinolone administration. A) the percentage of macrophages (F4/80+, CD68+) in the spleen was similar between saline and triamcinolone treated animals, while percentage of macrophages in the synovium (b) were decreased in triamcinolone group. c-d) spleen weight was significantly reduced in triamcinolone treated animals; the graph and pictures show spleens of groups sacrificed 3 days after triamcinolone/saline administration.

FIG. 6: Addition of empty capsids in 2 different ratios (5:1 and 20:1) improves intra-articular rAAV5-luciferase expression. Healthy mice (n=7 per group) were injected with rAAV5.CMV.Fluc (1.5e10 vg) intra-articularly, in 2 groups empty AAV5 capsids were added in different ratios. Luciferase expression was measured weekly until mice were sacrificed after 4 weeks. (a) Luminescence at week 4 is shown per group as averages; error bars, SEM. * P<0.05 and ** P<0.01 in groups with empty capsid addition versus the control group that only received genome containing vector (one-tailed unpaired t test).

FIG. 7: Luciferase expression in air pouch model of synovial inflammation (APSI). rAAV5.CMV.Fluc (3.16e11 vg) was administered into the air pouch of mice (n=5) on d0, d11, or d18 following air pouch formation. In the triamcinolone treated group, triamcinolone was administered (5 mg/kg) by i.m. injection on d9, followed by vector administration on d11. On d30, all mice were sacrificed and air pouch membranes were removed and subjected to luciferase assay. Luciferase expression per group is shown as averages with SEM. Black horizontal line indicates the limit of luciferase detection. The only groups that showed any detectable luciferase expression are when vector was injected on d0, or when triamcinolone was administered 2 days prior to vector.

FIG. 8: Effect of empty capsid and triamcinolone on intra-articular AAV5 gene expression in healthy mice. Healthy mice (n=17 per group, 34 total injected joints) were injected with rAAV5-CMV-Fluc intra-articularly (1.26e10 vg/joint)+/−empty AAV5 capsid (5:1 empty to full ratio) preceded 2 days prior with i.m. administration of either saline (NaCl) or triamcinolone. Luciferase expression was measured weekly by IVIS up to week 8. a) luciferase measurement over time for all groups. b) Luciferase expression in all groups at week 8. Data show is average per group+SEM. * p<0.05, ** p<0.01, *** p<0.001 as determined by one-tailed Mann Whitney test.

FIG. 9: Effect of empty capsid and triamcinolone on intra-articular AAV2 gene expression in healthy mice. Healthy mice (n=17 per group, 34 total injected joints) were injected with rAAV2-CMV-Fluc intra-articularly (1.26e10 vg/joint)+/−empty AAV2 capsid (5:1 empty ratio to full) preceded 2 days prior with i.m. administration of either saline (NaCl) or triamcinolone. Luciferase expression was measured weekly by IVIS up to week 4. a) luciferase measurement over time for all groups. b) Luciferase expression in all groups at week 4. Data show is average per group+SEM. ** p<0.01, *** p<0.001 as determined by one-tailed Mann Whitney test.

FIG. 10: AAV2 vs AA5 capid (VP1) alignment. The sequences show 57% identity as indicated by underscored amino acids.

The invention is further explained in the following examples. These examples do not limit the scope of the invention, but merely serve to clarify the invention.

### EXAMPLES

Methods

Vector Production and Empty Capsids

A rAAV5-transgene vector (Examples 1-7) or rAAV2-trangene vector (Example 8) was produced coding for Firefly Luciferase (Fluc) with a cytomegalovirus (CMV) promoter (rAAV5.CMV.Fluc; Children's Hospital of Philadelphia, Philadelphia, Pa.) as described previously. [Matsushita et al; Gene Ther 1998: 5; 938] In brief, the plasmid encodes the Fluc gene under the control of the CMV promoter and a human growth hormone polyadenylation signal. The transgene cassette is flanked by AAV-2 inverted terminal repeats and is packaged in capsid from AAV5.[Gao G P et al; PNAS 2002; 11854] The genome containing vector and empty AAV capsid particles were purified by combined chromatography and cesium chloride density gradient centrifugation. [Ayuso E, Mingozzi F et al; Gene Ther 2010: 17; 503] Vector titers were determined by qPCR and expressed as vector genomes/ml (vg/ml).

In Vivo Imaging Experiments in Mice

Intra-articular rAAV5 expression was investigated in male DBA mice (8-12 week old; Harlan Sprague Dawley, Horst, The Netherlands) in 5 animal experiments. Mice were injected with rAAV5.CMV.Fluc in both knee joints (with or without injection of ankle joints) and monitored periodically for luciferase expression (from 5 days up till 6 months). For animals without arthritis the vector was administered on day 1, in arthritic animals the vector was injected on day 17 or 24 after immunization, at the onset of disease. Animals received between 1.26e10 and 1.5e10 vg per knee joint (in a volume of 5 μl) and 0.75e10 vg per ankle joint (in a volume of 2.5 μl). Empty capsids were co-administered with the genome containing particles in several groups in a 5:1 or 20:1 ratio (ratio expressed as empty capsids to genome containing vectors). Groups consisted of 5 to 18 animals.

Collagen Induced Arthritis

Collagen induced arthritis (CIA) was induced by means of an intradermal injection of 100 μl collagen type II (2 mg/ml), diluted 1:1 in CFA (mineral oil and heat-killed M. Tuberculosis 2 mg/ml) (Chondrex Inc., Redmond, Wash., USA). On day 21 a booster injection was administered intraperitoneally containing 100 μg collagen type II dissolved in 100 μl NaCl.

Arthritis activity was scored 3-weekly from day 18 on with a semi-quantitative scoring system, each mouse paw was analyzed separately (0—normal; 1—swelling and/or erythema of 1 joint; 2—swelling in multiple joints; 3—swelling of all joints; 4—swelling of whole paw and at least one of the following symptoms: ankylosis, loss of function).

Air Pouch Synovial Inflammation (APSI) Model

Air pouches were formed as previously described (O'Boyle et al. (2009) FASEB J. 23 (11): 3906-3916). Briefly, 3 mls of air was injected subcutaneously into the back of each animal. The air pouches were kept inflated by re-injection of air as necessary. rAAV5.CMV.Fluc vector (3.16e11 vg) was administered into the pouch in a volume of 1 ml on d0, d11, or d18 post air pouch formation. For triamcinolone treated animals, triamcinolone was administered (5 mg/kg) by i.m. injection 2 days prior to vector administration on d11. On d30, mice were sacrificed and air pouch membrane was removed and snap frozen. Frozen air pouch tissue was homogenized in passive lysis buffer (Promega) and luciferase was measured by standard luciferase assay (Promega).

Macrophage Inhibition

Two days prior to vector administration, triamcinolone was administered intra-muscularly (i.m.), similar to the use in RA patients. RA patients receive triamcinolone in a dose of 0.4 to 1.0 mg per kg of bodyweight. Taking into account the faster metabolic rate in mice (factor 12.5), a dose of 5 mg/kg bodyweight was used, administered in a volume of 50 μl. Control groups received an i.m. injection with 50 μl NaCl. In a fourth experiment, i.m. triamcinolone was compared to intra-articular (i.a.) administration two days prior to vector administration, in a comparable dose of (in a volume of 5 μl). A control group received an i.a. injection with 5 μl NaCl.

Imaging of Luciferase Expression

Luciferase expression was measured at different time points after vector administration, from day 3 up till 6 months in different experiments. D-luciferin potassium-salt substrate (Caliper Life Sciences, Hopkinton, Mass., USA) was injected intraperitoneally (150 mg/kg of body weight, in a volume of approximately 200 μl). Photon counts were acquired 10 minutes after substrate administration for 5 minutes using a cooled charge-coupled device (CCD) camera system (Photon Imager, Biospace Lab, Paris, France). Light surface images were obtained immediately after each photon counting session to provide an anatomical view of the animal. Image processing and signal intensity quantification and analysis were performed using M3 Vision (Biospace Lab). Images were displayed as a pseudo-color photon count image, superimposed on a gray scale anatomic white-light image, allowing assessment of both bioluminescence intensity and its anatomical source. Regions of interest (ROI) were defined by drawing an elliptical ROI over the knee joint region. The surface area of the ROI was kept constant. The number of photons emitted per second per square centimetre per steradian was calculated as a measure of luciferase activity.

General Animal Conditions and Ethics Statement

Immunization, intra-articular injections and in vivo imaging were performed under isoflurane anaesthesia (3% isoflurane and oxygen). At the end of the experiments, animals were sacrificed by cardiac puncture under isoflurane anaesthesia, followed by cervical dislocation, after which hind paws, blood, lymph nodes and spleen were collected. The studies were reviewed and approved by the animal care and use committee of the University of Amsterdam (Amsterdam, The Netherlands; Permit Numbers: ART 102881, ART 102656, ART 102793, ART 102948, and ART 103021) and carried out in strict accordance with the recommendations in the Dutch Law on Animal Welfare (Wet op Dierproeven). Animals were maintained under pathogen-free conditions in the animal facility of the University of Amsterdam.

FLOW cytometry

Macrophages in spleen and synovium were analyzed by FLOW cytometry. Briefly, synovial cells were extracted by scraping cells from the joint followed by digestion with Liberase/DNase for 30 min at 37° C. Cells were then washed (PBS/EDTA) and passed through a cell strainer. Synovial cells were centrifuged (1400 rpm, 5 min, 4° C.) and resuspended in FACS buffer (PBS+1% BSA). Due to the low number of cells in the synovium, all animals from each group were pooled. Spleen cells were isolated by mechanical disruption and flushing cells through a cell strainer. Red blood cells were lysed by addition of RBC lysis buffer (Life Technologies), followed by 10 min incubation on ice. Cells were centrifuged and resuspended in FACS buffer. Cells (pooled synovium or splenocytes (1e6 cells) were blocked with 5% normal mouse serum (Sanquin) and stained with F4/80-APC and CD68-FITC labeled antibodies (BD Biosciences). Data was acquired on a BD Canto2 and was analyzed using FlowJo software (FLOWJO LLC, Ashland Oreg.)

Statistical Analysis

Luminescence over time was investigated using generalized estimating equations (GEE) to allow for longitudinal analysis (including all available longitudinal data and allowing unequal numbers of repeated measurements) (Twisk (2004) Eur. J. Epidemiol. 19(8):769-776). All other statistics were analyzed using Graphpad Prism (Ja Jolla, Calif., USA). For all tests, differences with a p-value of <0.05 were considered significant.

### Example 1

Inflammation Affects Intra-Articular rAAV5 Transgene Expression

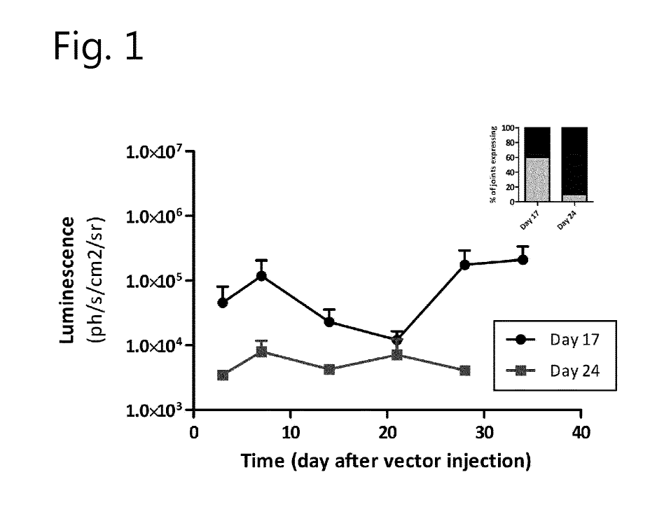

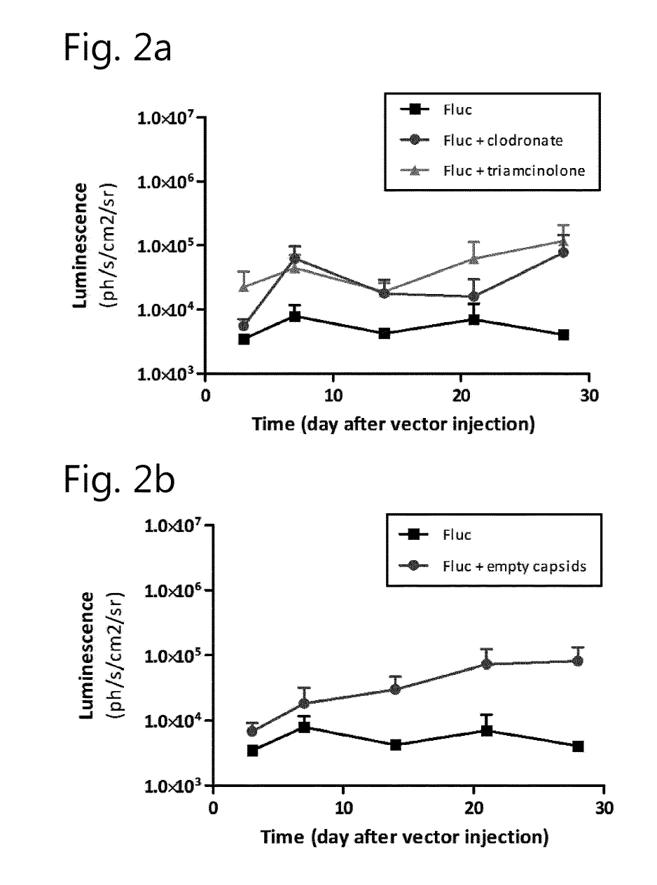

Fibroblast-like synoviocytes (FLS) are known to increase significantly in the inflamed joint of RA patients (Bartok and Firestein, Immunol Rev, 2010). This is also true for mouse models of RA, including the collagen induced arthritis (CIA) model. As FLS are the primary target cells for AAV5 in the joint, we hypothesized that administration of rAAV5-transgene vector after the onset of inflammation in the CIA model would lead to higher expression, due to a higher number of transduced FLS. To test this hypothesis, we administered an rAAV5-transgene vector encoding the Firefly luciferase gene (rAAV5.CMV.Fluc) intra-articularly in mice with CIA before (d17) or after (d24) the onset of arthritis. Surprisingly, this experiment showed that administration of a rAAV5-transgene vector after the onset of inflammation (day 24) resulted in lower expression per joint as well as a lower percentage of joints expressing the transgene, compared to vector administration before the onset of inflammation (day 17) (FIG. 1).

### Example 2

Immunosuppressive Agents Improve rAAV5 Transgene Expression

An explanation for decreased expression in animals with inflamed joints could be degradation or neutralization of the vector before it is able to transduce the target cells. During inflammation, there is not only an increase in the number of FLS, but there is also an increase in the number and activation of macrophages, thus we hypothesized that the decreased expression could be due to vector neutralization by macrophages (for example through phagocytosis or opsonization by soluble factors (complement)). To investigate this possibility we studied whether administration of agents that influence macrophage activity/number had an effect on rAAV5-transgene expression. Triamcinolone, a glucocorticosteroid, acts by inhibiting the activation and proliferation of macrophages.[Fauci A S, Dale D C, Balow J E; Ann Intern Med 1976; 84; 304-15] It is a pharmacological agent that is commonly used in humans, for example to treat acute inflammation in the joints of patients with RA. Systemic administration of glucocorticosteroids is also known to exert a local effect by decreasing the number and activity of macrophages in synovial tissue of RA patients. [Gerlag D M et al; Arthritis Rheum 2004; 50(12): 3783] A second agent used to deplete macrophages were clodronate containing liposomes. [van Roijen and Hendrikx, Methods in Molecular Medicine (605) pg 189-203, 2010]. The two agents were administered in separate groups 48 hours before vector administration.

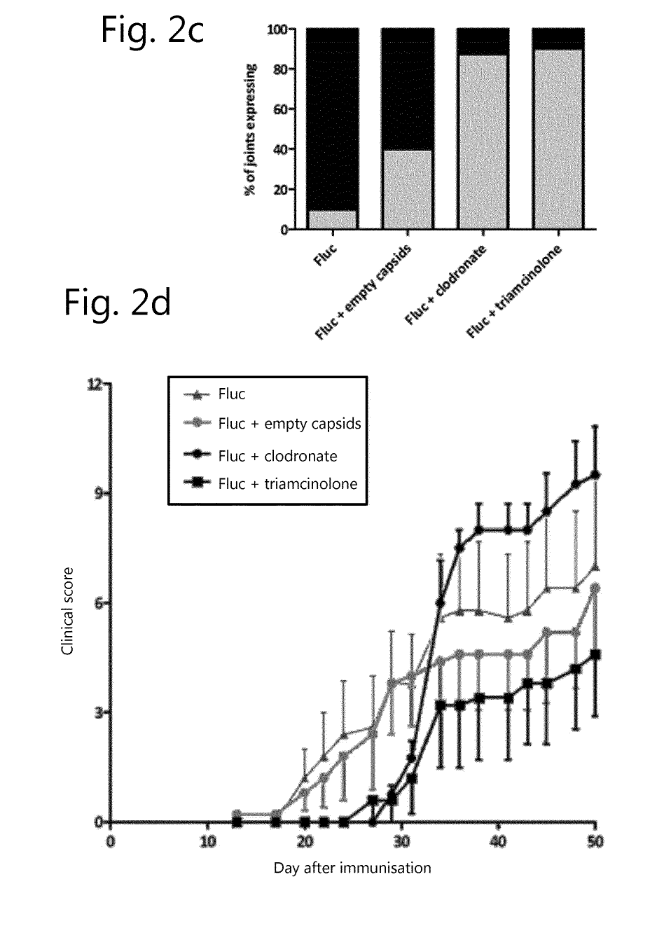

Both triamcinolone and liposomal clodronate improved rAAV5.CMV.Fluc expression over a period of 4 weeks, showing that either depleting or inhibiting macrophages led to an increase in gene expression (FIG. 2a).

We hypothesized that another way to avoid macrophage vector neutralization could be to add empty capsid particles upon vector administration. These empty capsids could be acting as a decoy to prevent degradation of the genome containing vector and therefore increasing the chances that full virus particles will be able to reach the target cells. When empty (AAV5) capsids were added to full genome containing capsids in a 5:1 ratio (empty to full), expression improved significantly (FIG. 2b). These data supported our hypothesis that the vector is likely being neutralized by macrophages. In all three groups also an increased percentage of positive joints was seen (FIG. 2c).

As triamcinolone is an anti-inflammatory agent, arthritis activity was closely monitored. Mice treated with triamcinolone showed a trend towards reduced arthritis activity (FIG. 2d).

### Example 3

Triamcinolone and Decoy Capsids have a Synergistic Effect on rAAV5-Transgene Expression

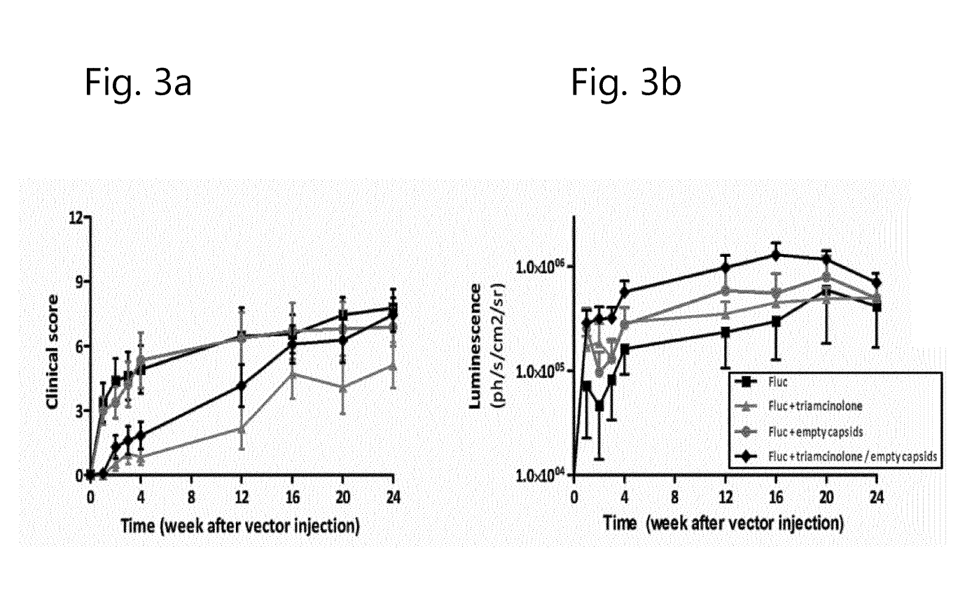

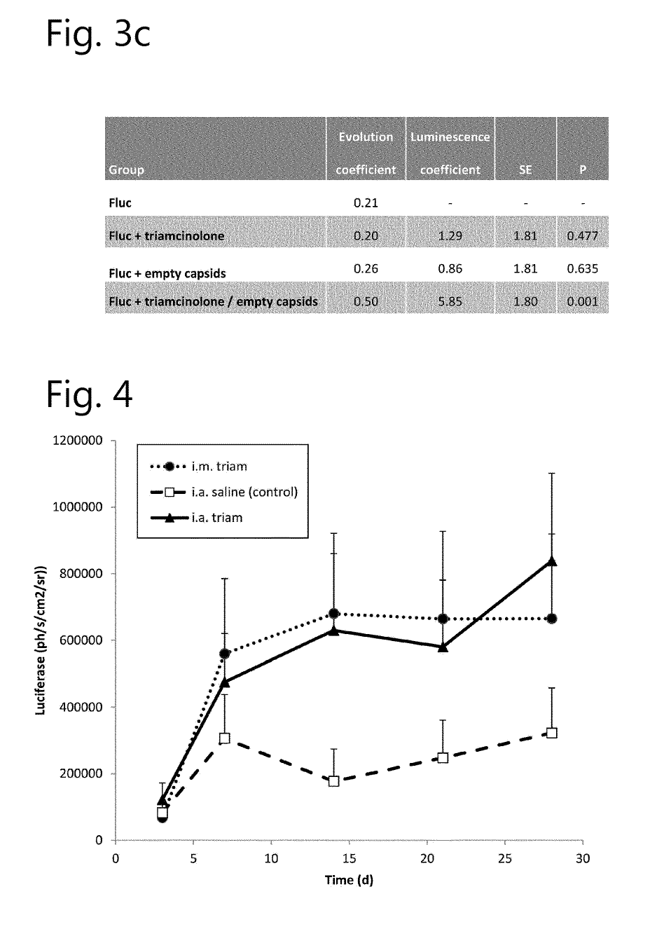

We then performed a long term follow up study to assess the duration of expression improved. The study showed that the combination of pharmacological inhibition and empty capsid decoy led to a synergistic enhancement of gene expression. As expected due to its anti-inflammatory effect, arthritis activity was lower in groups treated with triamcinolone up till week 4 (FIG. 3a). Long term arthritis activity was comparable between groups. Luciferase expression was monitored over time for a period of 6 months, remaining stable after an initial increase up to 1 month (FIG. 3b). The effect of addition of triamcinolone and/or empty capsids was analysed longitudinally using generalized estimating equations (GEE), allowing us to include all available longitudinal data and allowing unequal numbers of repeated measurements. A significant increase in luminescence was observed when both compounds were added (ratio of 5.85; p=0.001) (FIG. 3c). Separately the compounds showed a trend towards increased expression (not significant). This shows that the combination of triamcinolone and empty capsid had a synergistic effect on increasing gene expression.

A similar level of expression was observed in healthy versus arthritic mice (data not shown). Due to technical problems the IVIS data were not available for time point 8 weeks. A total of 15 animals (2-4 per group) was sacrificed prior to the end of the experiment due to reaching a humane endpoint.

As intra-articular administration of triamcinolone is a standard of care in RA patients, we wanted to determine if the route of triamcinolone administration (systemic vs local) had any effect on efficacy. To investigate this, mice (n=18) were administered triamcinolone locally (intra-articular (i.a.))(or saline as control), or systemically (intra-muscular (i.m.)) 2 days prior to i.a. administration of a composition of rAAV5.CMV.Fluc vector and empty capsids in a ratio of empty capsids:rAAV5.CMV.Fluc vector of 5:1. As can be seen in FIG. 4, pre-treatment of animals with triamcinolone resulted in an enhancement of gene expression. This was true whether the triamcinolone was administered systemically (i.m.) or locally (i.a.), indicating that the route of triamcinolone administration is not a critical factor for efficacy.

### Example 4

Triamcinolone has Differential Effects on Macrophages in Spleen v.s. Synovium

To further investigate the mechanism of action behind the effect of triamcinolone on transgene expression, an ex vivo analysis was carried out on synovial tissue and splenocytes to assess the effect on the numbers and activity of macrophages and other cell types. Cell populations of the different tissues were compared between triamcinolone and NaCl treated animals by FACS analysis 48 hrs after triamcinolone administration.

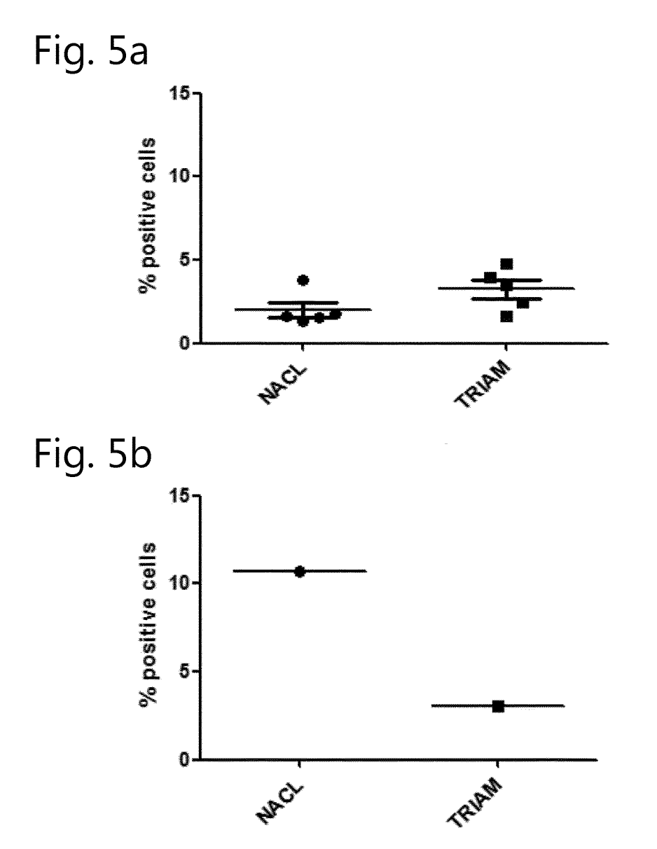

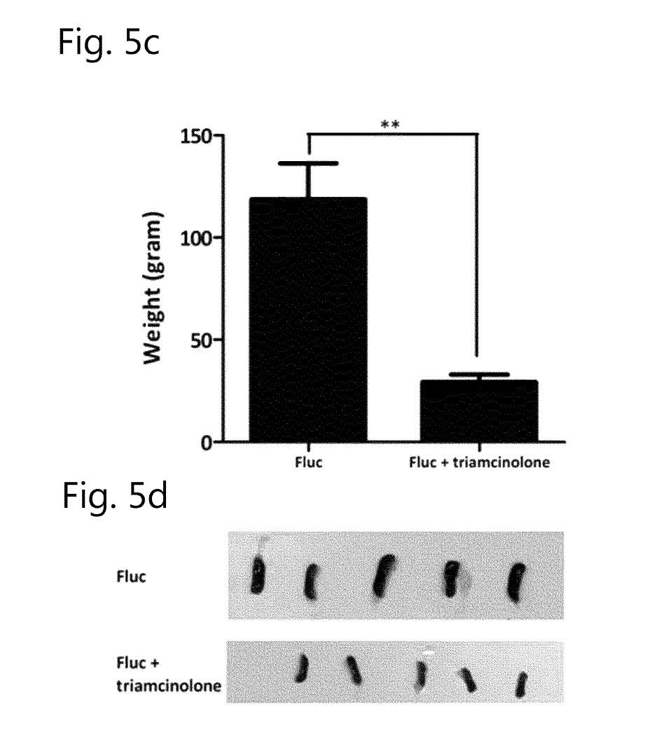

Remarkably, while the relative percentages of macrophages (CD68+, F4-80+) in the spleen remained similar in the triamcinolone treated animals remained (compared with saline) (FIG. 5a), absolute volume of spleen was decreased (4 fold) after triamcinolone treatment (p=0.0011) (FIGS. 5 c and d) at d25, with a reduced difference by day 29. In contrast with the spleen, the percentage of macrophages in the synovium was decreased after triamcinolone treatment (FIG. 5b). Note that given the small numbers of cells that can be extracted from the synovium, we had to pool all of the animals in a group in order to get enough cell counts.

### Example 5

AAV Empty Capsids Improve Transgene Expression in the Absence of Inflammation and Pre-Existing Humoral Immunity

All previous experiments were all performed in CIA models, in which animals experienced significant inflammation at the joint at the time of vector administration. We then decided to investigate whether the enhancement in luciferase expression could also be seen in healthy joints.

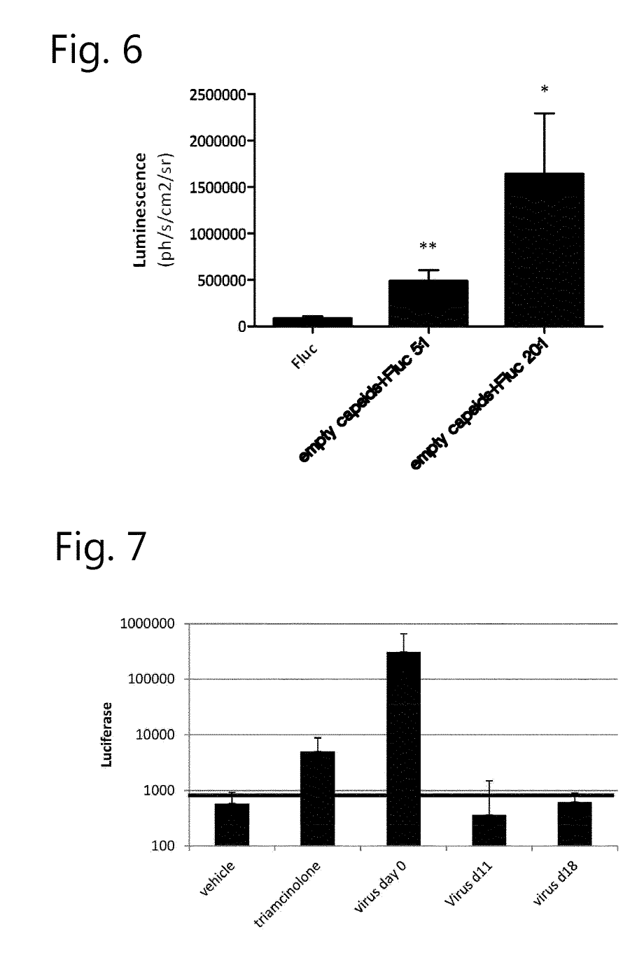

When empty capsids were added to genome containing particles in 2 different ratios, i.e. in a ratio of empty capsids to rAAV5.CMV.Fluc vector of 5:1 and 20:1, respectively, we observed a dose dependent increase in luminescence (FIG. 6). From day 3 after injection luciferase, expression was increased after addition of empty capsids to genome containing vector in a dose dependent manner (FIG. 6). The increase was 4.8 fold on average in animals injected with empty capsids in a 5:1 ratio to full capsids (p<0.01). A 20:1 ratio (empty to full) improved expression up till 20 fold (P<0.05).

### Example 6

Avoiding/Inhibiting Macrophages Allows for Expression in Air Pouch Synovial Inflammation (APSI) Model

The air pouch synovial inflammation (APSI) model was initially developed as a way to model human synovium in a mouse. It involves the injection of air under the skin on the back of a mouse. After 6-7 days, a lining membrane will form around this air pouch. This lining is very similar to the synovial lining that forms around the joint cavity, consisting primarily of fibroblast like cells and macrophages. When AAV expressing luciferase was administered into the air pouch on d7 (after formation of air pouch lining), we failed to see any expression, even at high vector doses (data not shown). We hypothesized that perhaps the macrophages lining the air pouch membrane were inhibiting the transduction of the vector (similar to what we have observed in intra-articular injected vector). We tested this hypothesis by either a) administering triamcinolone 2 days prior to vector administration or b) administering vector at d0, prior to the infiltration of macrophages into the air pouch lining. As can be seen in FIG. 7, luciferase expression was only observed when macrophages were either inhibited (e.g., triamcinolone) or were avoided (administration on d0). These data further support the hypothesis that macrophages are detrimental to AAV transduction and that strategies to avoid/inhibit AAV neutralization by macrophages are desired.

### Example 7

Combination of Empty Capsid and Triamcinolone Enhances Intra-Articular AAV5 Gene Expression

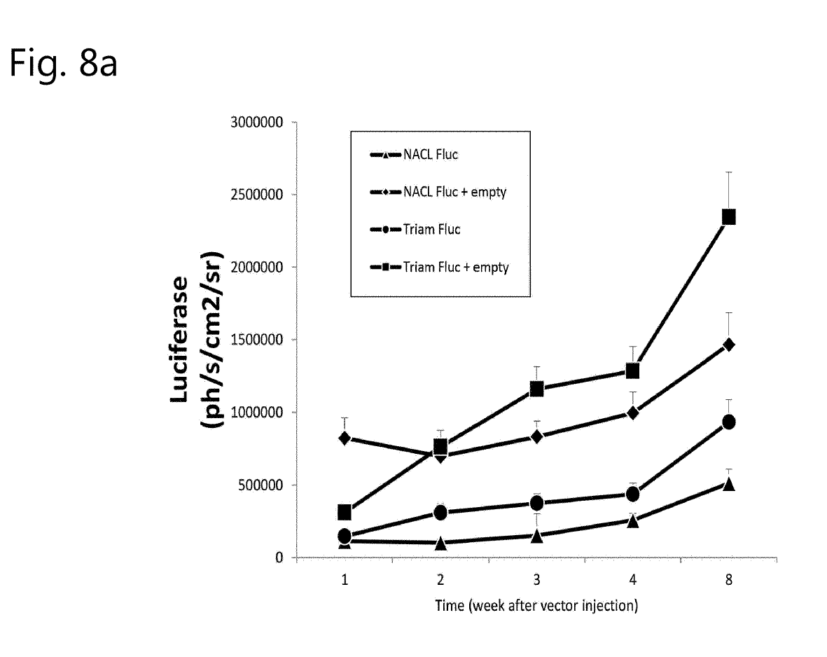

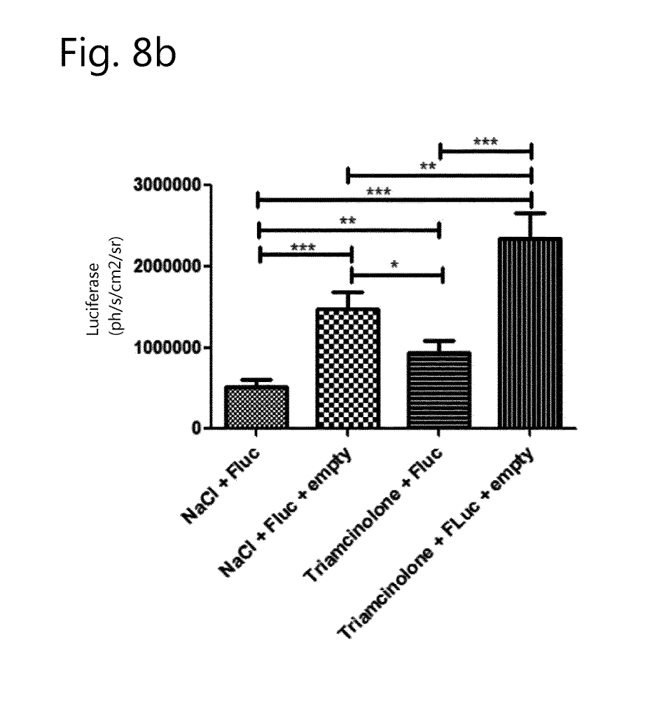

As we have shown that the combination of empty decoy capsid and triamcinolone was effective in increasing gene expression in the inflamed joint of mice with CIA (Example 3), and we have shown that empty capsid alone can increase intra-articular gene expression in healthy joints (Example 5), we wanted to determine if the combination of empty decoy capsid and triamcinolone would enhance gene expression even in the absence of inflammation (healthy joints). To determine this, groups of mice (n=18) were administered triamcinolone (5 mg/kg) or saline (control) by i.m. administration (50 μL total volume). Groups were then administered AAV5-CMV-Fluc (1.26e10 vg/joint) or AAV5-CMV-Fluc+empty AAV5 capsid (empty:full ratio=5:1) by intra-articular injection into both knees (5 μL total volume). Luciferase expression was monitored by live animal IVIS imaging. As can be seen in FIG. 8, both empty decoy capsid and triamcinolone treated animals showed increased gene expression compared with vector alone animals. The combination of empty capsid and triamcinolone gave rise to the highest increase in gene expression levels, similar to what was observed in inflamed (CIA) joints. These data indicate that the combination of triamcinolone and empty capsid are not only effective for increasing expression in diseased joints, but can also increase expression in healthy joints. These results support the hypothesis that the high number of macrophages in the synovial lining (even in healthy joints) are inhibiting AAV mediated expression, and that either adding decoy capsid particles and/or inhibiting macrophage activity can overcome this inhibition, leading to increased gene expression.

### Example 8

Combination of Empty Capsid and Triamcinolone is not Specific for AAV5, but Enhances Intra-Articular Gene Expression from Other Serotypes.

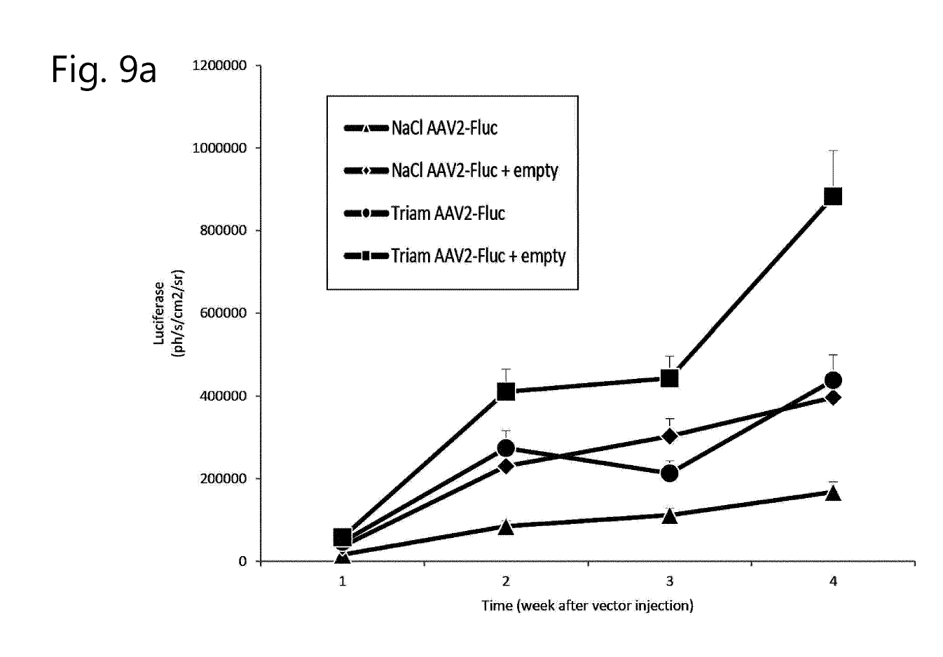

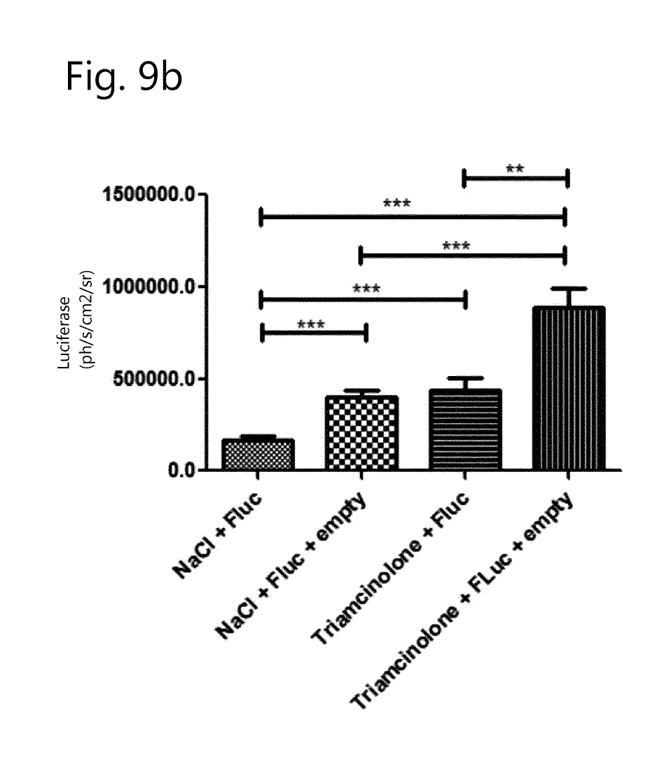

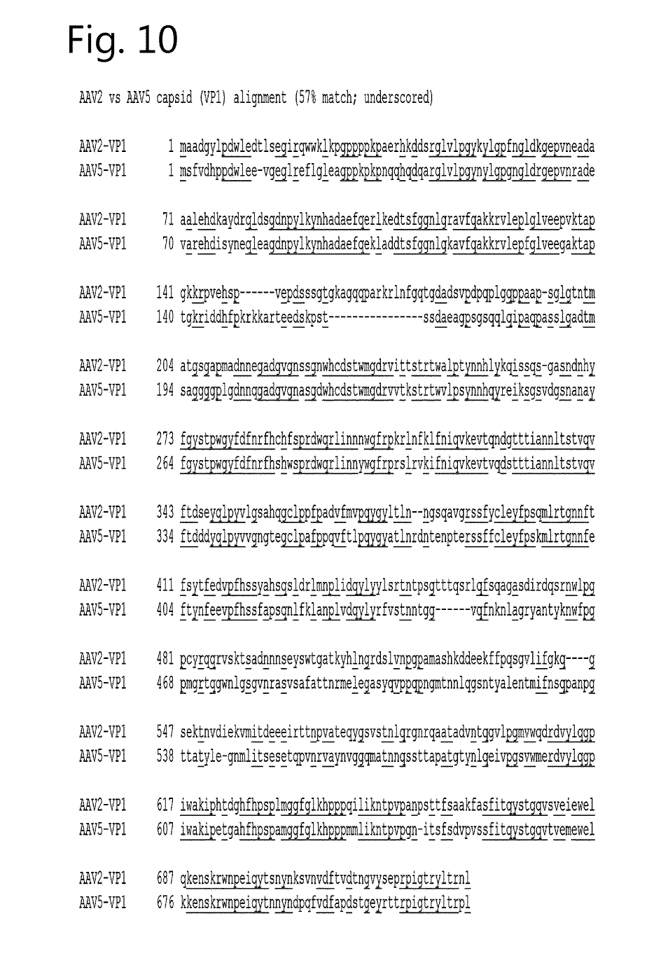

Our studies thus far have focused on AAV5 as this serotype has excellent tropism for the joint, however we hypothesize that macrophage neutralization of AAV is not serotype specific. This is because AAV update by macrophages is a general phenomenon utilizing scavenger receptors, and thus should not be limited to any one serotype, or any virus type whatsoever as macrophages are known to take up a wide range of viruses and bacteria. To test this hypothesis, we performed an experiment were we evaluated if triamcinolone and empty capsid could enhance gene expression from a serotype that is very different from AAV5, that being AAV2. AAV5 and AAV2 share only 57% homology at the amino acid level (see FIG. 10), making them two of the most diverse serotypes of AAV known. To determine this, an experiment identical to Example 7 was performed, however instead of using AAV5, we used an AAV2 vector. Groups of mice (n=18) were administered triamcinolone (5 mg/kg) or saline (control) by i.m. administration (50 μL total volume). Groups were then administered AAV2-CMV-Fluc (1.26e10 vg/joint) or AAV2-CMV-Fluc+empty AAV2 capsid (empty:full ratio=5:1) by intra-articular injection into both knees (54 total volume). Luciferase expression was monitored by live animal IVIS imaging. As can be seen in FIG. 9, similar to results seen with AAV5, both empty decoy capsid and triamcinolone treated animals showed increased gene expression compared with vector alone animals following AAV2 administration. The combination of empty capsid and triamcinolone gave rise to the highest increase in gene expression levels, similar to what was observed AAV5 treated animals. These data indicate that the combination of triamcinolone and empty capsid are not only effective for increasing expression with AAV5, but is also effective for other diverse serotypes, such as AAV2. Thus, it is clear that that the enhancement of gene expression by macrophage inhibition is not limited to AAV5, but is applicable to all AAV serotypes.

Given that empty AAV2 and AAV5 capsid were both able to increase gene expression, it follows that this enhancement of expression is not specific for a specific capsid serotype. We therefore conclude that the serotype of the empty capsid need not be of the same serotype as the full genome containing vector and any empty capsid serotype (natural or mutant) should be able to enhance intra-articular expression from any other AAV vector serotype (natural or mutant).

1. A rAAV vector composition and an immunosuppressant for use in a treatment comprising gene therapy, wherein the treatment comprises the administration of the rAAV vector composition and the administration of the immunosuppressant to an individual, wherein the rAAV vector composition comprises a rAAV-transgene vector and an empty capsid in a ratio of empty capsid to rAAV-transgene vector of at least 1:1.

2. A rAAV vector composition and an immunosuppressant for use in a treatment according to claim 1, wherein the at least one of the rAAV vector composition and immunosuppressant is administered locally.

3. A rAAV vector composition and an immunosuppressant for use in a treatment according to claim 1, wherein at least one of the rAAV vector composition and the immunosuppressant is administered systemically.

4. A rAAV vector composition and an immunosuppressant for use in a treatment according to claim 1, wherein the rAAV vector composition and the immunosuppressant are administered sequentially.

5. A rAAV vector composition and an immunosuppressant for use in a treatment according to claim 1, wherein the immunosuppressant is an innate immune cell inhibitor, a cytostatic drug, a non-steroidal anti-inflammatory drug, and/or an immunosuppressant biological such as a macrophage depleting antibody, a TNF blocker, IL-6 blocker and/or an IL-2 blocker and/or a purinergic signaling pathway modifying drug.

6. A rAAV vector composition and an immunosuppressant for use in a treatment according to claim 5, wherein the immunosuppressant is an innate immune cell inhibitor such as glucocorticoid and/or a liposomal bisphosphonate.

7. A rAAV vector composition and an immunosuppressant for use in a treatment according to claim 1, wherein the transgene comprised in the rAAV-transgene vector encodes a therapeutic protein.

8. A rAAV vector composition and an immunosuppressant for use in a treatment according to claim 1, wherein the gene therapy is for preventing, delaying, curing, reverting and/or treating an inflammatory condition or inflammatory disease.

9. A rAAV vector composition and an immunosuppressant for use in a treatment according to claim 8, wherein the transgene encodes a therapeutic anti-inflammatory protein.

10. A rAAV vector composition and an immunosuppressant for use in a treatment according to claim 8, wherein the inflammatory condition or disease is a rheumatic condition or disease.

11. A rAAV vector composition and an immunosuppressant for use in a treatment according to claim 2, wherein the rAAV vector composition is administered intra-articularly.

12. A rAAV vector composition and an immunosuppressant for use in a treatment according to claim 1, wherein the gene therapy is for treating, preventing, delaying, curing, reverting and/or treating an non-inflammatory condition or non-inflammatory disease.

13. A rAAV vector composition and an immunosuppressant for use in a treatment according to claim 1, wherein the rAAV vector composition further comprises a pharmaceutically acceptable carrier, diluents, solubilizer, filler, preservative and/or excipient.

14. A rAAV vector composition and an immunosuppressant for use in a treatment according to claim 1, wherein the immunosuppressant is comprised within the rAAV vector composition.

15. A rAAV vector composition, wherein the immunosuppressant is comprised within the rAAV vector composition.

16. A kit of parts comprising:
a rAAV vector composition comprises a rAAV-transgene vector and an empty capsid in a ratio of empty capsid to rAAV-transgene vector of at least 1:1 and;
an immunosuppressant.


SEQUENCE LISTING (19 sequences)

SEQ ID NO: 1 - DNA, 145 bp, Artificial
  single stranded AAV2 ITR, 5′ ITR
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcct

SEQ ID NO: 2 - DNA, 142 bp, Artificial
  single stranded AAV2 ITR, 3′ITR
aggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggg

SEQ ID NO: 3 - DNA, 95 bp, Artificial
  double stranded AAV2 ITR, 5′ITR
ctgcgcgctcgctcgctcactgaggccgcccgggcgtcgggcgacctttggtcgcccggcctcagtgagcgagcgagcgcgcagagagggagtgg

SEQ ID NO: 4 - DNA, 142 bp, Artificial; identical to SEQ ID NO: 2
  double stranded AAV2 ITR, 3′ ITR

SEQ ID NO: 5 - DNA, 167 bp, Artificial
  AAV5 ITR 5′ITR
ctctcccccctgtcgcgttcgctcgctcgctggctcgtttgggggggtggcagctcaaagagctgccagacgacggccctctggccgtcgcccccccaaacgagccagcgagcgagcgaacgcgacaggggggagagtgccacactctcaagcaagggggttttgta

SEQ ID NO: 6 - DNA, 167 bp, Artificial
  AAV5 ITR 3′ITR
tacaaaacctccttgcttgagagtgtggcactctcccccctgtcgcgttcgctcgctcgctggctcgtttgggggggtggcagctcaaagagctgccagacgacggccctctggccgtcgcccccccaaacgagccagcgagcgagcgaacgcgacaggggggagag

SEQ ID NO: 7 - DNA, 2208 bp, Artificial
  AAV2 Capsid
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctgactcgtaatctgtaa

SEQ ID NO: 8 - protein, 735 aa, Artificial [sequence omitted]

SEQ ID NO: 9 - protein, 598 aa, Artificial
  AAV2 VP2
TAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNRQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 10 - protein, 533 aa, Artificial
  AAV2 VP3
MATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNRQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 11 - DNA, 2190 bp, Artificial
  AAV5 capsid
atgtcttttgttgatcaccctccagattggttggaagaagttggtgaaggtcttcgcgagtttttgggccttgaagcgggcccaccgaaaccaaaacccaatcagcagcatcaagatcaagcccgtggtcttgtgctgcctggttataactatctcggacccggaaacggtctcgatcgaggagagcctgtcaacagggcagacgaggtcgcgcgagagcacgacatctcgtacaacgagcagcttgaggcgggagacaacccctacctcaagtacaaccacgcggacgccgagtttcaggagaagctcgccgacgacacatccttcgggggaaacctcggaaaggcagtctttcaggccaagaaaagggttctcgaaccttttggcctggttgaagagggtgctaagacgtttaaacgcgatcgcgcccctaccggaaagcggatagacgaccactttccaaaaagaaagaaggctcggaccgaagaggactccaagccttccacctcgtcagacgccgaagctggacccagcggatcccagcagctgcaaatcccagcccaaccagcctcaagtttgggagctgatacaatgtctgcgggaggtggcggcccattgggcgacaataaccaaggtgccgatggagtgggcaatgcctcgggagattggcattgcgattccacgtggatgggggacagagtcgtcaccaagtccacccgaacctgggtgctgcccagctacaacaaccaccagtaccgagagatcaaaagcggctccgtcgacggaagcaacgccaacgcctactttggatacagcaccccctgggggtactttgactttaaccgcttccacagccactggagcccccgagactggcaaagactcatcaacaactactggggcttcagaccccggtccctcagagtcaaaatcttcaacattcaagtcaaagaggtcacggtgcaggactccaccaccaccatcgccaacaacctcacctccaccgtccaagtgtttacggacgacgactaccagctgccctacgtcgtcggcaacgggaccgagggatgcctgccggccttccctccgcaggtctttacgctgccgcagtacggttacgcgacgctgaaccgcgacaacacagaaaatcccaccgagaggagcagcttcttctgcctagagtactttcccagcaagatgctgagaacgggcaacaactttgagtttacctacaactttgaggaggtgcccttccactccagcttcgctcccagtcagaacctcttcaagctggccaacccgctggtggaccagtacttgtaccgcttcgtgagcacaaataacactggcggagtccagttcaacaagaacctggccgggagatacgccaacacctacaaaaactggttcccggggcccatgggccgaacccagggctggaacctgggctccggggtcaaccgcgccagtgtcagcgccttcgccacgaccaataggatggagctcgagggcgcgagttaccaggtgcccccgcagccgaacggcatgaccaacaacctccagggcagcaacacctatgccctggagaacactatgatcttcaacagccagccggcgaacccgggcaccaccgccacgtacctcgagggcaacatgctcatcaccagcgagagcgagacgcagccggtgaaccgcgtggcgtacaacgtcggcgggcagatggccaccaacaaccagagctccaccactgcccccgcgaccggcacgtacaacctccaggaaatcgtgcccggcagcgtgtggatggagagggacgtgtacctccaaggacccatctgggccaagatcccagagacgggggcgcactttcacccctctccggccatgggcggattcggactcaaacacccaccgcccatgatgctcatcaagaacacgcctgtgcccggaaatatcaccagcttctcggacgtgcccgtcagcagcttcatcacccagtacagcaccgggcaggtcaccgtggagatggagtgggagctcaagaaggaaaactccaagaggtggaacccagagatccagtacacaaacaactacaacgacccccagtttgtggactttgccccggacagcaccggggaatacagaaccaccagacctatcggaacccgataccttacccgacccctttaa

SEQ ID NO: 12 - protein, 729 aa, Artificial
  AAV5 VP1
MSFVDHPPDWLEEVGEGLREFLGLEAGPPKPKPNQQHQDQARGLVLPGYNYLGPGNGLDRGEPVNRADEVAREHDISYNEQLEAGDNPYLKYNHADAEFQEKLADDTSFGGNLGKAVFQAKKRVLEPFGLVEEGAKTFKRDRAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLYRFVSTNNTGGVQFNKNLAGRYANTYKNWFPGPMGRTQGWNLGSGVNRASVSAFATTNRMELEGASYQVPPQPNGMTNNLQGSNTYALENTMIFNSQPANPGTTATYLEGNMLITSESETQPVNRVAYNVGGQMATNNQSSTTAPATGTYNLQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTNNYNDPQFVDFAPDSTGEYRTTRPIGTRYLTRPL

SEQ ID NO: 13 - protein, 593 aa, Artificial
  AAV5 VP2
TFKRDRAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLYRFVSTNNTGGVQFNKNLAGRYANTYKNWFPGPMGRTQGWNLGSGVNRASVSAFATTNRMELEGASYQVPPQPNGMTNNLQGSNTYALENTMIFNSQPANPGTTATYLEGNMLITSESETQPVNRVAYNVGGQMATNNQSSTTAPATGTYNLQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTNNYNDPQFVDFAPDSTGEYRTTRPIGTRYLTRPL

SEQ ID NO: 14 - protein, 532 aa, Artificial
  AAV5 VP3
MSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLYRFVSTNNTGGVQFNKNLAGRYANTYKNWFPGPMGRTQGWNLGSGVNRASVSAFATTNRMELEGASYQVPPQPNGMTNNLQGSNTYALENTMIFNSQPANPGTTATYLEGNMLITSESETQPVNRVAYNVGGQMATNNQSSTTAPATGTYNLQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTNNYNDPQFVDFAPDSTGEYRTTRPIGTRYLTRPL

SEQ ID NO: 15 - DNA, 2217 bp, Artificial
  AAV8 capsid
atggctgccgatggttatcttccagattggctcgaggacaacctctctgagggcattcgcgagtggtgggcgctgaaacctggagccccgaagcccaaagccaaccagcaaaagcaggacgacggccggggtctggtgcttcctggctacaagtacctcggacccttcaacggactcgacaagggggagcccgtcaacgcggcggacgcagcggccctcgagcacgacaaggcctacgaccagcagctgcaggcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaagagaccggtagagccatcaccccagcgttctccagactcctctacgggcatcggcaagaaaggccaacagcccgccagaaaaagactcaattttggtcagactggcgactcagagtcagttccagaccctcaacctctcggagaacctccagcagcgccctctggtgtgggacctaatacaatggctgcaggcggtggcgcaccaatggcagacaataacgaaggcgccgacggagtgggtagttcctcgggaaattggcattgcgattccacatggctgggcgacagagtcatcaccaccagcacccgaacctgggccctgcccacctacaacaaccacctctacaagcaaatctccaacgggacatcgggaggagccaccaacgacaacacctacttcggctacagcaccccctgggggtattttgactttaacagattccactgccacttttcaccacgtgactggcagcgactcatcaacaacaactggggattccggcccaagagactcagcttcaagctcttcaacatccaggtcaaggaggtcacgcagaatgaaggcaccaagaccatcgccaataacctcaccagcaccatccaggtgtttacggactcggagtaccagctgccgtacgttctcggctctgcccaccagggctgcctgcctccgttcccggcggacgtgttcatgattccccagtacggctacctaacactcaacaacggtagtcaggccgtgggacgctcctccttctactgcctggaatactttccttcgcagatgctgagaaccggcaacaacttccagtttacttacaccttcgaggacgtgcctttccacagcagctacgcccacagccagagcttggaccggctgatgaatcctctgattgaccagtacctgtactacttgtctcggactcaaacaacaggaggcacggcaaatacgcagactctgggcttcagccaaggtgggcctaatacaatggccaatcaggcaaagaactggctgccaggaccctgttaccgccaacaacgcgtctcaacgacaaccgggcaaaacaacaatagcaactttgcctggactgctgggaccaaataccatctgaatggaagaaattcattggctaatcctggcatcgctatggcaacacacaaagacgacgaggagcgtttttttcccagtaacgggatcctgatttttggcaaacaaaatgctgccagagacaatgcggattacagcgatgtcatgctcaccagcgaggaagaaatcaaaaccactaaccctgtggctacagaggaatacggtatcgtggcagataacttgcagcagcaaaacacggctcctcaaattggaactgtcaacagccagggggccttacccggtatggtctggcagaaccgggacgtgtacctgcagggtcccatctgggccaagattcctcacacggacggcaacttccacccgtctccgctgatgggcggctttggcctgaaacatcctccgcctcagatcctgatcaagaacacgcctgtacctgcggatcctccgaccaccttcaaccagtcaaagctgaactctttcatcacgcaatacagcaccggacaggtcagcgtggaaattgaatgggagctgcagaaggaaaacagcaagcgctggaaccccgagatccagtacacctccaactactacaaatctacaagtgtggactttgctgttaatacagaaggcgtgtactctgaaccccgccccattggcacccgttacctcacccgtaatctgtaa

SEQ ID NO: 16 - protein, 782 aa, Artificial
  AAV8 VP1
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPLGEPPAAPSGVGPNTMAAGGGAPMADNNEGADGVGSSSGNWHCDSTWLGDRVITTSTRTWALPTYNNHLYKQISNGTSGGATNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQTTGGTANTQTLGFSQGGPNTMANQAKNWLPGPCYRQQRVSTTTGQNNNSNFAWTAGTKYHLNGRNSLANPGIAMATHKDDEERFFPSNGILIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNLMAADGYLPDWLEDNLSEGIREWWALKPGAPKPKANQQKQDDGRG

SEQ ID NO: 17 - protein, 601 aa, Artificial
  AAV8 VP2
TAPGKKRPVEPSPQRSPDSSTGIGKKGQQPARKRLNFGQTGDSESVPDPQPLGEPPAAPSGVGPNTMAAGGGAPMADNNEGADGVGSSSGNWHCDSTWLGDRVITTSTRTWALPTYNNHLYKQISNGTSGGATNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQTTGGTANTQTLGFSQGGPNTMANQAKNWLPGPCYRQQRVSTTTGQNNNSNFAWTAGTKYHLNGRNSLANPGIAMATHKDDEERFFPSNGILIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 18 - protein, 535 aa, Artificial
  AAV8 VP3
MAAGGGAPMADNNEGADGVGSSSGNWHCDSTWLGDRVITTSTRTWALPTYNNHLYKQISNGTSGGATNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQTTGGTANTQTLGFSQGGPNTMANQAKNWLPGPCYRQQRVSTTTGQNNNSNFAWTAGTKYHLNGRNSLANPGIAMATHKDDEERFFPSNGILIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 19 - DNA, 166 bp, Artificial
  promoter
ctagcaggcctcgaggggactttccacaaggggactttccgtcgaggggactttccacaaggggactttccgtcgaggggactttccacaaggggactttccgtcgaggcctgtaggcgtgtacggtgggaggcttatataagcagagctcaagcttggtaccgag